SEQUENCE LISTING (16 sequences)

SEQ ID NO: 1 - protein, 18 aa, synthetic construct
MVKGKHHHHHHNGKGKSK

SEQ ID NO: 2 - protein, 88 aa, synthetic construct
MVKGKHHHHHHNGKGKSKGPRTLCGAELVDALQFVCGDRGFYFNKPTGYGSSSRRAPQTGIVDECCFRSCDLRRLEMYCAPLKPAKSA

SEQ ID NO: 3 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 4 - protein, 71 aa, synthetic construct
FVNQHLCGSHLVEALYLVCGDRGFYFNKPTGYGSSSRRAPQTGIVDECCFRSCDLRRLEMYCAPLKPAKSA

SEQ ID NO: 5 - protein, 83 aa, synthetic construct
MFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 6 - protein, 70 aa, Synthetic construct
GPRTLCGAELVDALQFVCGDRGFYFNKPTGYGSSSRRAPQTGIVDECCFRSCDLRRLEMYCAPLKPAKSA

SEQ ID NO: 7 - protein, 67 aa, synthetic construct
TLCGAELVDALQFVCGDRGFYFNKPTGYGSSSRRAPQTGIVDECCFRSCDLRRLEMYCAPLKPAKSA

SEQ ID NO: 8 - protein, 83 aa, synthetic construct
MFPAMPLSSLFVNGPETLCGAELVDALQFVCGDRGFYFNKPTGYGSSSRRAPQTGIVDECCFRSCDLRRLEMYCAPLKPAKSA

SEQ ID NO: 9 - protein, 83 aa, synthetic construct
MFPAMPLSSLFVNGPGTLCGAELVDALQFVCGDRGFYFNKPTGYGSSSRRAPQTGIVDECCFRSCDLRRLEMYCAPLKPAKSA

SEQ ID NO: 10 - protein, 21 aa, Homo sapiens
GIVEQCCTSICSLYQLENYCN

SEQ ID NO: 11 - protein, 30 aa, Homo sapiens
FVNQHLCGSHLVEALYLVCGERGFFYTPKT

SEQ ID NO: 12 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 13 - protein, 462 aa, synthetic construct
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFHAFVNQHLCGSHLVEALYLVCGDRGFYFNKPTGYGSSSRRAPQTGIVDECCFRSCDLRRLEMYCAPLKPAKSA

SEQ ID NO: 14 - protein, 318 aa, synthetic construct
DKGDGWILGEPSVVSSQILNPNETGTFSQSLTKSKEVSINVNFSVGFTSEFIQASVEYGFGITIGEQNTIERSVSTTAGPNEYVYYKVYATYRKYQAIRISHGNISDDGSIYKLTGIWLSKTSADSLGNIDQGSLIETGERCVLTVPSTDIEKEILDLAAATERLNLTDALNSNPAGNLYDWRSSNSYPWTQKLNLHLTITATGQKYRILASKIVDFNIYSNNFNNLVKLEQSLGDGVKDHYVDIHAFVNQHLCGSHLVEALYLVCGDRGFYFNKPTGYGSSSRRAPQTGIVDECCFRSCDLRRLEMYCAPLKPAKSA

SEQ ID NO: 15 - protein, 275 aa, synthetic construct
MHHHHHHGDDDDKDKGDGWILGEPSVVSSQILNPNETGTFSQSLTKSKEVSINVNFSVGFTSEFIQASVEYGFGITIGEQNTIERSVSTTAGPNEYVYYKVYATYRKYQAIRISHGNISDDGSIYKLTGIWLSKTSADSLGNIDQGSLIETGERCVLTVPSTDIEKEILDLAAATERLNLTDALNSNPAGNLMFPAMPLSSLFVNGPRTLCGAELVDALQFVCGDRGFYFNKPTGYGSSSRRAPQTGIVDECCFRSCDLRRLEMYCAPLKPAKSA

SEQ ID NO: 16 - protein, 472 aa, synthetic construct
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTHAMFPAMPLSSLFVNGPRTLCGAELVDALQFVCGDRGFYFNKPTGYGSSSRRAPQTGIVDECCFRSCDLRRLEMYCAPLKPAKSA

(19) United States
(12) Patent Application Publication
(10) Pub. No.: US 2025/0367267 A1
(43) Pub. Date: Dec. 4, 2025

(54) TREATING AUTOIMMUNE DISEASES WITH INSULIN-LIKE GROWTH FACTOR 1 RECEPTOR LIGAND CONJUGATED TO AN AGENT

(71) Applicant: Lirum Therapeutics, Inc., New York, NY (US)

(72) Inventor: Matthew HOBERMAN, Demarest, NJ (US)

(21) Appl. No.: 18/876,854

(22) PCT Filed: Jun. 29, 2023
(86) PCT No.: PCT/US2023/069425

§ 371 Date: Dec. 19, 2024

Related U.S. Application Data
(60) Provisional application No. 63/367,240, filed on Jun. 29, 2022.

(51) Int. Cl. A61K 38/30; A61K 31/519; A61K 38/16; A61K 38/28; A61K 38/46; A61K 45/06; A61P 37/00; G01N 15/14; G01N 33/53

(52) U.S. Cl. CPC A61K 38/30; A61K 31/519; A61K 38/164; A61K 38/28; A61K 38/465; A61K 45/06; A61P 37/00; G01N 15/14; G01N 33/53; G01N 2474/20

(57) ABSTRACT

The subject matter described herein provides methods for treating an autoimmune disease in a subject, comprising administering to the subject an effective amount of a conjugate that comprises an IGF-1R ligand, or portion or variant thereof, and a disease-modifying agent.

### CROSS REFERENCE TO RELATED APPLICATIONS

This application claims the benefit of U.S. Provisional Application No. 63/367,240, filed on Jun. 29, 2022.

### FIELD

The presently disclosed subject matter relates generally to methods of treating autoimmune diseases in particular by administering an IGF-1R ligand conjugated to a cytotoxic agent.

### REFERENCE TO A SEQUENCE LISTING

The Sequence Listing written in file name 597947SEQLIST.xml is 17 kilobytes, was created on Jun. 29, 2023, and is hereby incorporated by reference.

### BACKGROUND

The human immune system is an intricate network of diverse cell types that has evolved to defend the host against pathogens and malignancies as well as maintain tissue homeostasis. Autoimmune diseases occur when the immune system is misdirected toward host tissues, frequently as a result of loss of B cell or T cell tolerance, and can range from organ-specific to systemic disease. Despite recent advances in research, autoimmune diseases remain poorly understood and difficult to treat and thus contribute substantially to morbidity and mortality each year.

There remains a need in the art for improved methods of treating autoimmune diseases.

### BRIEF SUMMARY

In accordance with the description, this application describes methods of treatment with an IGF-1R ligand conjugated to a disease-modifying agent.

In one embodiment, the present application provides a method for treating an autoimmune disease in a subject in need thereof comprising administering to the subject an effective amount of a conjugate, wherein said conjugate comprises an IGF-1R ligand and a disease-modifying agent.

In some embodiments, the autoimmune disease is selected from: adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, Cushing's syndrome, cutaneous graft-versus-host disease (GVHD), dense deposit disease, dermatomyositis, essential mixed cryoglobulinemia, fibromyalgia-fibromyositis, glomerulonephritis (e.g., IgA nephropathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, inflammatory arthritis, irritable bowel disease, juvenile arthritis, lichen planus, lichen sclerosus, lupus [e.g., systemic lupus erythematosus (SLE), cutaneous lupus, discoid lupus], Ménière's disease, mixed connective tissue disease, morphea, multiple sclerosis, Myasthenia Gravis, myocarditis, type 1 or immune-mediated diabetes mellitus, neuritis, other endocrine gland failure, pemphigus vulgaris, pernicious anemia, polyarteritis nodosa, polychondritis, polyendocrinopathies, polyglandular syndromes, polymyalgia rheumatica, polymyositis, post-MI, primary agammaglobulinemia, primary biliary cirrhosis, psoriasis, psoriatic arthritis, Raynaud's phenomenon, relapsing polychondritis, Reiter's syndrome, rheumatic heart disease, rheumatoid arthritis, sarcoidosis, Sjogren's syndrome, stiff-man syndrome, systemic sclerosis (SSc), takayasu arteritis, temporal arteritis/giant cell arteritis, ulcerative colitis, urticaria, uveitis, Uveitis Ophthalmia, vasculitides such as dermatitis herpetiformis vasculitis, vitiligo, and Wegener's granulomatosis.

In a specific embodiment, the autoimmune disease is rheumatoid arthritis, Graves' disease, SSc, Cushing's syndrome, idiopathic pulmonary fibrosis, SLE, or Crohn's disease. In another specific embodiment, the SSc is diffuse cutaneous SSc.

In some embodiments, the treatment results in an alteration or impairment in the function of IGF-1R-expressing cells in said subject. In other embodiments, the treatment results in a reduction in the number of IGF-1R-expressing cells in the subject. In a specific embodiment, the reduction is caused by killing of IGF-1R-expressing cells. Without being bound by theory, it is believed that simple inhibition of the IGF-1R pathway may be insufficient as a fully curative treatment due to signaling by other, potentially redundant, pathways that can continue to act as disease effectors or by compensatory mechanisms or pathways that are upregulated and can lead to adaptive resistance. Thus, the targeted delivery of an agent that is disease-modifying, including one that results in the killing of IGF-1R-expressing cells, may provide benefit beyond (including being preferential over) simple IGF-1R pathway inhibition, as it bypasses the effects of all such potentially redundant and/or compensatory pathways.

In another specific embodiment, the IGF-1R-expressing cells are B lymphocytes and/or T lymphocytes.

In another embodiment, the IGF-1R is overexpressed by cells in the subject relative to cells from a healthy subject or a subject that has not been diagnosed with an autoimmune disease.

In another embodiment, the frequency of cells expressing IGF-1R in the subject is increased relative to cells from a healthy subject or a subject that has not been diagnosed with an autoimmune disease. In another embodiment, the frequency of IGF-1R-expressing cells is measured by flow cytometry or immunohistochemistry.

In some embodiments, the treatment results in disease modification in said subject.

In some embodiments, the conjugate comprises wildtype insulin-like growth factor 1 (IGF-1) (SEQ ID NO:3), wildtype insulin (SEQ ID NO:10 and SEQ ID NO:11), or wildtype insulin-like growth factor 2 (IGF-2) (SEQ ID NO:12). In some embodiments, the conjugate comprises a variant of wildtype IGF-1 (SEQ ID NO:3), a variant of wildtype insulin (SEQ ID NO:10 and SEQ ID NO:11), or a variant of wildtype IGF-2 (SEQ ID NO:12). In some embodiments, the variant of wildtype IGF1 is at least 90% identical to IGF-1 (SEQ ID NO:3), the variant of wildtype insulin is at least 90% identical to insulin (SEQ ID NO:10 and SEQ ID NO:11), or the variant of wildtype IGF-2 is at least 90% identical to IGF-2 (SEQ ID NO:12).

In some embodiments, the conjugate comprises a variant of IGF-1 that has reduced binding affinity for IGF binding proteins (IGFBPs) as compared to wildtype IGF-1 (SEQ ID NO:3)) or a variant of IGF-2 that has reduced binding affinity for IGF binding proteins as compared to wildtype IGF-2 (SEQ ID NO:12). In some embodiments, the variant of IGF-1 has increased affinity for the IGF-1R than wildtype IGF-1 or the variant of IGF-2 has increased affinity for the IGF-1R than wildtype IGF-2. In a specific embodiment, the conjugate comprises 765IGF (SEQ ID NO:2), IGF132 (SEQ ID NO:4), long-R3-IGF-1 (SEQ ID NO:5), R3-IGF-1 (SEQ ID NO:6), des (1-3)-IGF-1 (SEQ ID NO:7), long-IGF-1 (SEQ ID NO:8), or long-G3-IGF-1 (SEQ ID NO:9).

In some embodiments, the IGF-1R ligand, or portion or variant thereof, comprises a leader sequence. In a specific embodiment, the leader sequence comprises SEQ ID NO:1.

In a specific embodiment, the conjugate comprises 765IGF (SEQ ID NO:2).

In some embodiments, the disease-modifying agent comprises a cytotoxic agent. In some embodiments, the cytotoxic agent comprises a chemotherapeutic agent. In a specific embodiment, the chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine. In another specific embodiment, the chemotherapeutic agent is methotrexate.

In some embodiments, the cytotoxic agent comprises a toxin. In a specific embodiment, the toxin is Clostridium perfringens enterotoxin, diphtheria toxin, ricin A chain, deglycosylated ricin A chain, Pseudomonas exotoxin, A chain toxins, ribosome inactivating proteins, a-sarcin, aspergillin, abrin, restrictocin, bacterial endotoxin, the lipid A moiety of bacterial endotoxin, cholera toxin, or a ribonuclease. In a specific embodiment, the toxin comprises Clostridium perfringens enterotoxin, or a portion of variant thereof. In a specific embodiment, the conjugate comprises SEQ ID NO:14 or SEQ ID NO:15. In other specific embodiments, the toxin comprises diphtheria toxin, or a portion or variant thereof. In a specific embodiment, the conjugate comprises SEQ ID NO:13 or SEQ ID NO:16.

In some embodiments, the subject having an autoimmune disease treated in accordance with the methods described herein has not previously received treatment for said autoimmune disease, has previously received treatment for said autoimmune disease, has relapsed from previous treatment for said autoimmune disease, or was refractory to previous treatment for said autoimmune disease.

In some embodiments, the conjugate is administered in combination with one or more other therapies. In a specific embodiment, the one or more other therapies comprises one or more of the following: prednisone, hydroxychloroquine, chloroquine, belimumab, anifrolumab, abatacept, atacicept, lupuzor, rituximab, voclosporin, aldesleukin, baricitinib, BIIB059, BI655064, bortezomib, BT063, cenerimod, dapirolizumab pegol, edratide, filgotinib, GS-9876, iberdomide, IFN-a kinoid, iguratimod, nelfinavir, obinutuzumab, OMS721, rapamycin, RC18, RSLV-132, SM101, theralizumab, ustekinumab, vobarilizumab, XmAb5871, blisibimod, tabalumab, epratuzumab, rigerimod, tacrolimus, rontalizumab, sifalimumab, anifrolumab, tocilizumab, infliximab, metelimumab, fresolimumab, rilonacept, cyclophosphamide, methotrexate, nintedanib, JBT-101, imatinib, pirfenidone, nilotinib, dasatinib, SAR100842, BMS-986202, BAY41-2272, riociguat, resunab, ixekizumab, brodalumab, tralokinumab, etanercept, adalimumab, golimumab, secukinumab, tildrakizumab, tofacitinib, and guselkumab.

In some embodiments, the conjugate is administered at a dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, 9.5, or 10.0 μEq/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 μEq/kg of body weight.

In some embodiments, the conjugate is administered at a dose of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, or 16.5 mg/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight.

In some embodiments, the conjugate is administered at a dose that is the maximum tolerated dose (MTD).

In some embodiments, the conjugate is administered daily, every other day, every three days, every four days, every five days, every six days, once per week, once every two weeks, once every three weeks, once every four weeks, once per month, every two months, or every three months.

In some embodiments, the conjugate is administered orally, intravenously, subcutaneously, intramuscularly, or topically.

In some embodiments, the method of the present invention does not cause unacceptable hyperglycemia in the subject.

In a specific embodiment, provided herein is a method for treating an autoimmune disease in a subject in need thereof by administering an effective amount of a conjugate, wherein said conjugate comprises 765IGF (SEQ ID NO:2) and methotrexate.

In another specific embodiment, provided herein is a method for using modulation in the frequency of IGF-1R-expressing cells to monitor the effectiveness of a treatment of an autoimmune disease in a subject in need thereof, the method comprising administering to the subject an effective amount of a conjugate, wherein said conjugate comprises (i) an IGF-1R ligand, or portion or variant thereof, and (ii) a disease-modifying agent, and subsequently determining the frequency of IGF-1R-expressing cells in a sample obtained from the subject.

Additional objects and advantages will be set forth in part in the description which follows, and in part will be obvious from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF FIGURES

FIGS. 1A-1E show the effect of LX-101 on bulk subpopulations of peripheral blood mononuclear cells (PBMCs) from patients with systemic lupus erythematosus (SLE), rheumatoid arthritis (RA), and systemic sclerosis (SSc) compared to a vehicle control. The percentage of live cells determined by flow cytometry is shown for B cell (FIG. 1A), CD4⁺ T cell (FIG. 1B), CD8⁺ T cell (FIG. 1C), NK cell (FIG. 1D), and NKT cell (FIG. 1E) subpopulations. Similar results were observed for vehicle control and LX-101 treated cells.

FIGS. 2A-2E show the effect of LX-101 on IGF-1R⁺ subpopulations of PBMCs from patients with systemic lupus erythematosus (SLE), rheumatoid arthritis (RA), and systemic sclerosis (SSc) compared to a vehicle control. The percentage of live IGF-1R⁺ cells is shown for B cell (FIG. 2A), CD4⁺ T cell (FIG. 2B), CD8⁺ T cell (FIG. 2C), NK cell (FIG. 2D), and NKT cell (FIG. 2E) subpopulations. Reduced percentages of live IGF-1R⁺ cells were observed for cells treated with LX-101 as compared to the vehicle control.

FIGS. 3A-3.J show flow cytometry dot plots of PBMCs from an SLE patient. The dot plots show expression levels of IGF-1R (CD221) in specific PBMC subpopulations. In FIGS. 3A, 3C, 3E, 3G, and 3I, cells were treated with a vehicle control. In FIGS. 3B, 3D, 3F, 3H, and 3J, cells were treated with 17 μM of LX-101. FIGS. 3A-3B show the CD4⁺ T cell population. FIGS. 3C-3D show the CD8⁺ T cell population. FIGS. 3E-3F show the CD19⁺ B cell population. FIGS. 3G-3H show the CD56⁺CD3⁺ NKT cell population. FIGS. 31-3J show the CD56⁺CD3⁻ NK cell population.

FIGS. 4A-4J show flow cytometry dot plots of PBMCs from an RA patient. The dot plots show expression levels of IGF-1R (CD221) in specific PBMC subpopulations. In FIGS. 4A, 4C, 4E, 4G, and 41, cells were treated with a vehicle control. In FIGS. 4B, 4D, 4F, 4H, and 4J, cells were treated with 17 μM of LX-101. FIGS. 4A-4B show the CD4⁺ T cell population. FIGS. 4C-4D show the CD8⁺ T cell population. FIGS. 4E-4F show the CD19⁺ B cell population. FIGS. 4G-4H show the CD56⁺CD3⁺ NKT cell population. FIGS. 4I-4J show the CD56⁺CD3⁻ NK cell population.

FIGS. 5A-5J show flow cytometry dot plots of PBMCs from an SSc patient. The dot plots show expression levels of IGF-1R (CD221) in specific PBMC subpopulations. In FIGS. 5A, 5C, 5E, 5G, and 5I, cells were treated with a vehicle control. In FIGS. 5B, 5D, 5F, 5H, and 5J, cells were treated with 17 μM of LX-101. FIGS. 5A-5B show the CD4⁺ T cell population. FIGS. 5C-5D show the CD8⁺ T cell population. FIGS. 5E-5F show the CD19⁺ B cell population. FIGS. 5G-5H show the CD56⁺CD3⁺ NKT cell population. FIGS. 5I-5J show the CD56⁺CD3⁻ NK cell population.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents and other references mentioned herein are incorporated by reference into their entirety.

### I. Definitions

As used herein, a patient or subject and the like is any mammal suffering from an autoimmune disease. As used herein, the term “mammal” includes, but is not limited to, humans, mice, rats, guinea pigs, monkeys, dogs, cats, horses, cows, pigs, and sheep.

As used herein, the term “conjugate” refers to a molecule comprising an IGF-1R ligand, or portion or variant thereof, and a disease-modifying agent.

As used herein, the term “cytotoxic agent” refers to any agent capable of preventing, delaying, reducing and/or reversing the activity, severity, and/or progression of the disease when treated in accordance with the methods described herein. Any suitable cytotoxic agent that results in cell killing can be used in the conjugate and in the method of treating an autoimmune disease.

As used herein, the term “residue” or “residue of” a chemical moiety or compound refers to a chemical moiety or compound that is bound to a molecule, whereby through the binding, at least one covalent bond has replaced at least one atom of the original chemical moiety or compound, resulting in a residue of the chemical moiety or compound in the molecule.

As used herein, a subject is “refractory” to prior treatment if the subject has failed to achieve a response to a therapy such that the therapy is determined to not be therapeutically effective, such as: failure to reach clinical endpoint, including any of response, extended duration of response, extended disease-free survival, relapse-free survival, and progression-free survival.

As used herein, “and/or” refers to and encompasses any and all possible combinations of one or more of the associated listed items, as well as the lack of combinations when interpreted in the alternative (“or”).

As used herein, the term “about,” when referring to a measurable value such as an amount of a compound or agent of the current subject matter, dose, time, temperature, and the like, is meant to encompass variations of ±20%, ±10%, ±5%, ±1%, ±0.5%, or even ±0.1% of the specified amount.

As used herein, conditional language used herein, such as, among others, “can,” “could,” “might,” “may,” “e.g.,” and the like, unless specifically stated otherwise or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Definitions of additional terms may be set forth below.

### II. Compositions

### Agents for Treatment

This application provides methods for treating an autoimmune disease in a subject in need thereof comprising administering to the subject an effective amount of a conjugate, wherein said conjugate comprises an IGF-1R ligand, or portion or variant thereof, and a disease-modifying agent.

In this or any of the embodiments of the present invention, the conjugate can comprise a chemical conjugate in which the IGF-1R ligand and the disease-modifying agent are chemically linked together, either directly or through a chemical linker. In other embodiments, the conjugate is a genetic recombinant in which the conjugate is expressed as a single polypeptide. When the conjugate is a recombinant conjugate, the translated conjugate preferably comprises a toxin, or portion or variant thereof, linked via a peptide bond to the IGF-1R ligand.

In certain embodiments, the conjugate is a fusion protein described in U.S. Pat. No. 9,675,671, which is hereby incorporated by reference in its entirety.

### IGF-1R Ligands

The IGF-1R is a heterotetramer consisting of two extracellular ligand-binding a subunits and two transmembrane b subunits with kinase activity that mediate signal transduction. The native ligands to the IGF-1R are IGF-1, IGF-2, and insulin. The IGF-1R has the highest affinity for IGF-1, followed by IGF-2, and can bind to insulin with 50- to 100-fold lower affinity. IGF-1R can also form hybrid receptors by dimerization with the insulin receptor. See Hakuno et al. J Mol Endocrinol. 61(1):T69-T86 (2018).

In certain embodiments, the IGF-1R ligand in the conjugate of the present invention comprises wildtype IGF-1 (SEQ ID NO:3), wildtype insulin (SEQ ID NO:10 and SEQ ID NO:11; mature insulin consists of two chains connected by disulfide bonds, chain A, corresponding to SEQ ID NO:10, and chain B, corresponding to SEQ ID NO:11, hence the recitation of two SEQ ID NOs), or wildtype IGF-2 (SEQ ID NO:12). In other embodiments, the IGF-1R ligand in the conjugate comprises a variant of wildtype IGF-1 (SEQ ID NO:3), a variant of wildtype insulin (SEQ ID NO:10 and SEQ ID NO:11), or a variant of wildtype IGF-2 (SEQ ID NO:12). In a specific embodiment, the variant of wildtype IGF-1 is at least 70%, 75%, 80%, 85%, 90%, 95%, or 99% identical to IGF-1 (SEQ ID NO:3), said variant of wildtype insulin is at least 70%, 75%, 80%, 85%, 90%, 95%, or 99% identical to insulin (SEQ ID NO:10 and SEQ ID NO:11), or said variant of wildtype IGF-2 is at least 70%, 75%, 80%, 85%, 90%, 95%, or 99% identical to IGF-2 (SEQ ID NO:12).

In another specific embodiment, the IGF-1R ligand in the conjugate comprises a variant of IGF-1 that has reduced binding affinity for IGFBPs as compared to wildtype IGF-1 (SEQ ID NO:3) or a variant of IGF-2 that has reduced binding affinity for IGFBPs as compared to wildtype IGF-2 (SEQ ID NO:12). IGFBPs belong to a family of at least six proteins that bind to IGF-1 and IGF-2 with high affinity. IGFBPs bind to the majority of IGFs in circulation, increasing their half-life, regulating their bioavailability, and generally inhibit their ability to bind to the IGF receptors. See Baxter, Am J Physiol Endocrinol Metab., 278(6):E967-76 (2000) and Allard et al. Front Endocrinol (Lausanne). 9; 9:117 (2018). Thus, variants of IGF-1 or IGF-2 that have reduced binding to IGFBPs have greater bioactivity in vivo.

IGF-1 variants with reduced binding affinity for IGFBPs are known in the art and include IGF132 (disclosed in U.S. Pat. No. 4,876,242), in which the first 17 amino acids of the B chain of insulin (SEQ ID NO:11) replace the first 16 amino acids of human IGF-1 (SEQ ID NO:3); R3-IGF-1 (SEQ ID NO:6), in which glutamic acid in position 3 of the native human IGF-1 (SEQ ID NO:3) is substituted by arginine; and des (1-3) IGF-1 (SEQ ID NO:7), which lacks the first three amino acids of human IGF-1 (SEQ ID NO:3). R3-IGF-1 and des (1-3) IGF-1 are described in Francis et al., J Mol Endocrinol. 8(3):213-23 (1992). In some embodiments, the conjugate comprises IGF132 (SEQ ID NO:4), R3-IGF-1 (SEQ ID NO:6), or des (1-3)-IGF-1 (SEQ ID NO:7).

In another embodiment, the variant of IGF-1 has higher affinity for the IGF-1R than wildtype IGF-1, or the variant of IGF-2 has higher affinity for the IGF-1R than wildtype IGF-2.

In another embodiment, the IGF-1R ligand, or portion or variant thereof, comprises a leader sequence. Leader sequences can incorporate tags, such as polyhistidine tags, to facilitate protein purification, as well as provide sites for conjugation of disease-modifying agents. In a specific embodiment, the leader sequence comprises SEQ ID NO:1.

In some embodiments, the IGF-1R ligand in the conjugate comprises 765IGF (SEQ ID NO:2), long-R3-IGF-1 (SEQ ID NO:5), long-IGF-1 (SEQ ID NO:8), or long-G3-IGF-1 (SEQ ID NO:9). 765IGF, long-R3-IGF-1, long-IGF-1, and long-G3-IGF-1 have N-terminal leader sequences that facilitate protein purification and provide sites for conjugation of disease-modifying agents, as described above. 765IGF (SEQ ID NO:2) comprises SEQ ID NO:1 followed by R3-IGF-1 (SEQ ID NO:6); long-R3-IGF-1 (SEQ ID NO:5) comprises the first 11 amino acids of methionyl porcine growth hormone, followed by a Val-Asn dipeptide, followed by R3-IGF-1 (SEQ ID NO:6); long-IGF-1 (SEQ ID NO:8) comprises the first 11 amino acids of methionyl porcine growth hormone, followed by a Val-Asn dipeptide, followed by human IGF-1 (SEQ ID NO:3); and long-G3-IGF-1 comprises the first 11 amino acids of methionyl porcine growth hormone, followed by a Val-Asn dipeptide, followed by a variant of human IGF-1 in which glutamic acid in position 3 of the native human IGF-1 (SEQ ID NO:3) is substituted by glycine. In some embodiments, the conjugate comprises 765IGF (SEQ ID NO:2).

### Disease-Modifying Agents

As used herein, the term disease-modifying agent refers to any agent capable of reducing and/or reversing the activity, severity, and/or progression of the disease when treated in accordance with the methods described in the present invention.

In a specific embodiment, the disease-modifying agent of the conjugate is a cytotoxic agent. Any suitable cytotoxic agent that results in cell killing can be used in the conjugate of the invention and in the method of treating an autoimmune disease.

In a specific embodiment, the cytotoxic agent is a chemotherapeutic agent. Any suitable chemotherapeutic agent that results in cell killing can be used in the conjugates of the invention and in the methods of treating an autoimmune disease. For instance, in particular embodiments, the chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine. In a specific embodiment, the chemotherapeutic agent is methotrexate.

In certain embodiments, a conjugate comprises more than one cytotoxic agent bound to the IGF-1R ligand. In certain aspects, the conjugate can comprise one to 12 cytotoxic agents, or 6 to 10 cytotoxic agents, or about 8 cytotoxic agents. In certain aspects, the conjugate can comprise one to twelve covalently bound cytotoxic agents, or 6 to 10 covalently bound cytotoxic agents, or about 8 covalently bound cytotoxic agents. In certain aspects, the cytotoxic agent(s) are covalently bound to any available position on the IGF-1R ligand. In certain aspects, the cytotoxic agent(s) are covalently bound to any available lysine residue. In certain aspects, the cytotoxic agent(s) are covalently bound to any available lysine in the leader sequence when present.

In certain embodiments, the subject matter described herein is directed to methods for treating an autoimmune disease in a subject, the method comprising administering to the subject a conjugate comprising an IGF-1R ligand, or portion or variant thereof, and a cytotoxic agent, wherein the IGF-1R ligand, or portion or variant thereof comprises SEQ ID NO:2, the cytotoxic agent is methotrexate, wherein the methotrexate is covalently bound to a lysine of SEQ ID NO:2, and the autoimmune disease is selected from the group consisting of rheumatoid arthritis, Graves' disease, SSc, Cushing's syndrome, idiopathic pulmonary fibrosis, SLE, and Crohn's disease. In a certain aspect of these embodiments, the conjugate is LX-101 (a conjugate as described above wherein the IGF-1R ligand is SEQ ID NO:2, the cytotoxic agent is methotrexate, and wherein at least 6 and up to 10, or at least 6 and up to 9, or at least 7 and up to 9, or at least 8 and up to 9 methotrexate residues are each covalently bound to a lysine residue of SEQ ID NO:2).

In certain embodiments, the number of methotrexate residues per conjugate is 6, 7, 8, 9 or 10. In certain embodiments, the average number of methotrexate residues per conjugate in a composition is 6, 7, 8, 9 or 10.

In other embodiments, the cytotoxic agent is a toxin. Any suitable toxin that results in cell killing can be used in the conjugates of the present invention and in the methods of treating an autoimmune disease. Toxins can be derived from plants, fungus, or bacteria. For instance, in particular embodiments, the toxin is Clostridium perfringens enterotoxin, diphtheria toxin, ricin A chain, deglycosylated ricin A chain, Pseudomonas exotoxin, A chain toxins, a ribosome inactivating proteins, α-sarcin, aspergillin, abrin, restrictocin, bacterial endotoxin, the lipid A moiety of bacterial endotoxin, cholera toxin, or a ribonuclease. In a specific embodiment, the toxin comprises Clostridium perfringens enterotoxin, or a portion of variant thereof. In a specific embodiment, the conjugate comprises SEQ ID NO:14 or SEQ ID NO:15. In other specific embodiments, the toxin comprises diphtheria toxin, or a portion or variant thereof. In a specific embodiment, the conjugate comprises SEQ ID NO:13 or SEQ ID NO:16.

Table 1 provides a listing of certain sequences referenced herein.

TABLE 1
Description of the Sequences

| Description | Sequences | SEQ ID NO |
|---|---|---|
| Leader sequence | MVKGKHHHHHHNGKGKSK | 1 |
| 765IGF | MVKGKHHHHHHNGKGKSKGPRTLCGAELVDALQFVC | 2 |
| | GDRGFYFNKPTGYGSSSRRAPQTGIVDECCFRSCDLRRL | |
| | EMYCAPLKPAKSA | |
| human IGF-1 | GPETLCGAELVDALQFVCGDRGFYFNKPTGYGSSSRRAP | 3 |
| | QTGIVDECCFRSCDLRRLEMYCAPLKPAKSA | |
| IGF132 | FVNQHLCGSHLVEALYLVCGDRGFYFNKPTGYGSSSRR | 4 |
| | APQTGIVDECCFRSCDLRRLEMYCAPLKPAKSA | |
| Long-R3-IGF-1 | MFPAMPLSSLFVNGPRTLCGAELVDALQFVCGDRGFYF | 5 |
| | NKPTGYGSSSRRAPQTGIVDECCFRSCDLRRLEMYCAPL | |
| | KPAKSA | |
| R3-IGF-1 | GPRTLCGAELVDALQFVCGDRGFYFNKPTGYGSSSRRA | 6 |
| | PQTGIVDECCFRSCDLRRLEMYCAPLKPAKSA | |
| des(1-3)IGF-1 | TLCGAELVDALQFVCGDRGFYFNKPTGYGSSSRRAPQT | 7 |
| | GIVDECCFRSCDLRRLEMYCAPLKPAKSA | |
| Long-IGF-1 | MFPAMPLSSLFVNGPETLCGAELVDALQFVCGDRGFYF | 8 |
| | NKPTGYGSSSRRAPQTGIVDECCFRSCDLRRLEMYCAPL | |
| | KPAKSA | |
| Long-G3-IGF-1 | MFPAMPLSSLFVNGPGTLCGAELVDALQFVCGDRGFYF | 9 |
| | NKPTGYGSSSRRAPQTGIVDECCFRSCDLRRLEMYCAPL | |
| | KPAKSA | |
| Human insulin | GIVEQCCTSICSLYQLENYCN | 10 |
| A-chain | | |
| Human insulin B- | FVNQHLCGSHLVEALYLVCGERGFFYTPKT | 11 |
| chain | | |
| Human IGF-2 | AYRPSETLCGGELVDTLQFVCGDRGFYFSRPASRVSRRS | 12 |
| | RGIVEECCFRSCDLALLETYCATPAKSE | |
| Diphtheria toxin- | GADDVVDSSKSFVMENFSSYHGTKPGYVDSIQKGIQKP | 13 |
| IGF132 fusion | KSGTQGNYDDDWKGFYSTDNKYDAAGYSVDNENPLSG | |
| | KAGGVVKVTYPGLTKVLALKVDNAETIKKELGLSLTEP | |
| | LMEQVGTEEFIKRFGDGASRVVLSLPFAEGSSSVEYINN | |
| | WEQAKALSVELEINFETRGKRGQDAMYEYMAQACAGN | |
| | RVRRSVGSSLSCINLDWDVIRDKTKTKIESLKEHGPIKNK | |
| | MSESPNKTVSEEKAKQYLEEFHQTALEHPELSELKTVTG | |
| | TNPVFAGANYAAWAVNVAQVIDSETADNLEKTTAALSI | |
| | LPGIGSVMGIADGAVHHNTEEIVAQSIALSSLMVAQAIPL | |
| | VGELVDIGFAAYNFVESIINLFQVVHNSYNRPAYSPGHK | |
| | TQPFHAFVNQHLCGSHLVEALYLVCGDRGFYFNKPTGY | |
| | GSSSRRAPQTGIVDECCFRSCDLRRLEMYCAPLKPAKSA | |
| Clostridium | DKGDGWILGEPSVVSSQILNPNETGTFSQSLTKSKEVSIN | 14 |
| perfringens | VNFSVGFTSEFIQASVEYGFGITIGEQNTIERSVSTTAGPN | |
| enterotoxin- | EYVYYKVYATYRKYQAIRISHGNISDDGSIYKLTGIWLS | |
| IGF132 fusion | KTSADSLGNIDQGSLIETGERCVLTVPSTDIEKEILDLAA | |
| | ATERLNLTDALNSNPAGNLYDWRSSNSYPWTQKLNLHL | |
| | TITATGQKYRILASKIVDFNIYSNNFNNLVKLEQSLGDGV | |
| | KDHYVDIHAFVNQHLCGSHLVEALYLVCGDRGFYFNKP | |
| | TGYGSSSRRAPQTGIVDECCFRSCDLRRLEMYCAPLKPA | |
| | KSA | |
| Clostridium | MHHHHHHGDDDDKDKGDGWILGEPSVVSSQILNPNET | 15 |
| perfringens | GTFSQSLTKSKEVSINVNFSVGFTSEFIQASVEYGFGITIG | |
| enterotoxin- | EQNTIERSVSTTAGPNEYVYYKVYATYRKYQAIRISHGN | |
| long-R3-IGF-1 | ISDDGSIYKLTGIWLSKTSADSLGNIDQGSLIETGERCVLT | |
| fusion | VPSTDIEKEILDLAAATERLNLTDALNSNPAGNLMFPAM | |
| | PLSSLFVNGPRTLCGAELVDALQFVCGDRGFYFNKPTGY | |
| | GSSSRRAPQTGIVDECCFRSCDLRRLEMYCAPLKPAKSA | |
| Diphtheria toxin- | MGADDVVDSSKSFVMENFSSYHGTKPGYVDSIQKGIQK | 16 |
| long-R3-IGF-1 | PKSGTQGNYDDDWKGFYSTDNKYDAAGYSVDNENPLS | |
| fusion | GKAGGVVKVTYPGLTKVLALKVDNAETIKKELGLSLTE | |
| | PLMEQVGTEEFIKRFGDGASRVVLSLPFAEGSSSVEYINN | |
| | WEQAKALSVELEINFETRGKRGQDAMYEYMAQACAGN | |
| | RVRRSVGSSLSCINLDWDVIRDKTKTKIESLKEHGPIKNK | |
| | MSESPNKTVSEEKAKQYLEEFHQTALEHPELSELKTVTG | |
| | TNPVFAGANYAAWAVNVAQVIDSETADNLEKTTAALSI | |
| | LPGIGSVMGIADGAVHHNTEEIVAQSIALSSLMVAQAIPL | |
| | VGELVDIGFAAYNFVESIINLFQVVHNSYNRPAYSPGHK | |
| | THAMFPAMPLSSLFVNGPRTLCGAELVDALQFVCGDRG | |
| | FYFNKPTGYGSSSRRAPQTGIVDECCFRSCDLRRLEMYC | |
| | APLKPAKSA | |

### Pharmaceutical Compositions

The conjugates of the present invention can be formulated in pharmaceutical compositions for use in the methods described herein. In some embodiments, the pharmaceutical composition comprises an effective amount of a conjugate of the invention and a pharmaceutically acceptable carrier or vehicle. Such pharmaceutical compositions can be formulated to be suitable for administration to a subject and can be in any form that allows for the composition to be administered to a subject.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of condition the subject has, the use of the composition as part of a multi-drug regimen, the particular form of the composition of the invention, and the manner of administration. The pharmaceutical compositions comprise an effective amount of a composition of the invention such that a suitable dosage will be obtained.

The term “carrier” refers to a diluent, adjuvant or excipient, with which a composition of the invention is administered. Any auxiliary, stabilizing, thickening, lubricating and coloring agents can be used. In one embodiment, when administered to a subject, the compositions of the invention and pharmaceutically acceptable carriers are sterile. Water may be a carrier when the composition of the invention is administered intravenously. Saline solutions and aqueous dextrose and glycerol solutions can also be employed as liquid carriers, particularly for injectable solutions. The present compositions, if desired, can also contain minor amounts of pH buffering agents.

The liquid compositions of the invention, whether they are solutions, suspensions, or other like form, can also include one or more of the following: sterile diluents such as water for injection, saline solution, physiological saline, Ringer's solution, isotonic sodium chloride, fixed oils such as synthetic mono or digylcerides which can serve as the solvent or suspending medium, polyethylene glycols, glycerin, cyclodextrin, propylene glycol, or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; agents for the adjustment of pH such as hydrochloric acid; and agents for the adjustment of tonicity such as sodium chloride or dextrose. A parenteral composition can be enclosed in an ampoule, a disposable syringe, or a multiple-dose vial made of glass, plastic or other material. In some embodiments, physiological saline is an adjuvant. An injectable composition may be sterile.

The present compositions can take the form of solutions, suspensions, tablets, pills, pellets, capsules, capsules containing liquids, powders, sustained-release formulations, suppositories, emulsions, aerosols, sprays, suspensions, or any other form suitable for use. Examples of suitable pharmaceutical carriers are described in Remington's Pharmaceutical Sciences by E. W. Martin.

In some embodiments, the compositions of the invention are formulated in accordance with routine procedures as a pharmaceutical composition adapted for intravenous administration to human subjects. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where a composition of the invention is to be administered by infusion, it can be dispensed, for example, with an infusion bottle containing sterile pharmaceutical grade water or saline. Where the composition of the invention is administered by injection, an ampoule of sterile water for injection or saline can be provided so that the ingredients can be mixed prior to administration.

The pharmaceutical compositions can be prepared using methodology well known in the pharmaceutical art. For example, a composition intended to be administered by injection can be prepared by combining a composition of the invention with water so as to form a solution. A surfactant can be added to facilitate the formation of a homogeneous solution or suspension. Surfactants are complexes that can non-covalently interact with a composition of the invention so as to facilitate dissolution or homogeneous suspension of the composition of the invention in the aqueous delivery system.

### III. Methods

### Methods for Producing IGF-1R Conjugates

Methods for producing conjugates of the present invention are known in the art.

The nucleotide sequences encoding the IGF-1R ligands can be produced by standard recombinant DNA techniques or by protein synthetic techniques, cloned into an appropriate expression vector using standard molecular biology techniques, expressed in bacterial, insect, or mammalian cells, and purified by any method known in the art for purification of a protein.

Conjugates of the present invention comprising an IGF-1R ligand and a chemotherapeutic agent can be made by standard chemistry and protein conjugation techniques and are described in U.S. Pat. Nos. 7,811,982; 9,675,671; and 9,801,923.

Conjugates of the present invention comprising an IGF-1R ligand and a toxin can be made as fusion proteins by standard recombinant DNA techniques and are described in U.S. Pat. No. 8,017,102.

### Methods of Treatment

Provided herein are methods of treating an autoimmune disease using a conjugate, wherein said conjugate comprises an IGF-1R ligand, or portion or variant thereof, and a disease-modifying agent.

Non-limiting examples of autoimmune diseases that can be treated by the methods of the present invention include adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, Cushing's syndrome, cutaneous graft-versus-host disease (GVHD), dense deposit disease, dermatomyositis, essential mixed cryoglobulinemia, fibromyalgia-fibromyositis, glomerulonephritis (e.g., IgA nephropathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, inflammatory arthritis, irritable bowel disease, juvenile arthritis, lichen planus, lichen sclerosus, lupus (e.g., SLE, cutaneous lupus, discoid lupus), Ménière's disease, mixed connective tissue disease, morphea, multiple sclerosis, Myasthenia Gravis, myocarditis, type 1 or immune-mediated diabetes mellitus, neuritis, other endocrine gland failure, pemphigus vulgaris, pernicious anemia, polyarteritis nodosa, polychondritis, polyendocrinopathies, polyglandular syndromes, polymyalgia rheumatica, polymyositis, post-MI, primary agammaglobulinemia, primary biliary cirrhosis, psoriasis, psoriatic arthritis, Raynaud's phenomenon, relapsing polychondritis, Reiter's syndrome, rheumatic heart disease, rheumatoid arthritis, sarcoidosis, Sjogren's syndrome, stiff-man syndrome, systemic sclerosis (SSc), takayasu arteritis, temporal arteritis/giant cell arteritis, ulcerative colitis, urticaria, uveitis, Uveitis Ophthalmia, vasculitides such as dermatitis herpetiformis vasculitis, vitiligo, and Wegener's granulomatosis.

In a specific embodiment, the present disclosure provides a method of treating rheumatoid arthritis, Graves' disease, SSc, Cushing's syndrome, idiopathic pulmonary fibrosis, SLE, or Crohn's disease in a subject in need thereof.

In another specific embodiment, the present disclosure provides a method of treating diffuse cutaneous SSc in a subject in need thereof.

### Methods of Measuring Clinical Efficacy

As used herein, the terms “subject” and “patient” are used interchangeably.

In some embodiments, the method for treating an autoimmune disease in a subject in need thereof with the conjugates of the present invention results in disease modification in the subject. As used herein, disease modification can refer to any prevention, delay, reduction, and/or reversal in the activity, severity, and/or progression of the disease in a subject when treated in accordance with the methods described in the present invention. Disease modification can result from direct or indirect effects on the underlying pathophysiology of the disease as a result of treatment with the conjugates of the present invention.

Methods of evaluating autoimmune patients to determine amelioration of disease symptoms or reduction in disease are known by those skilled in the art. For example, amelioration of disease symptoms or reduction in disease may be assessed via a disease activity score or index [e.g., Disease Activity Score 28 (DAS28) in rheumatoid arthritis; SLE Disease Activity Index (SLEDAI) in SLE]. See van Riel et al., Clin Exp Rheumatol, 34(5 Suppl 101):S40-S44 (2016) and Ohmura, Mod Rheumatol, 31(1):20-28 (2021). The effects of a therapy can be monitored by continual assessment of a disease activity score throughout the course of treatment by comparison of the score at a later time point to the score at a prior time point (e.g., before administration of therapy). Biomarkers that predict clinical benefit (e.g., rheumatoid factor and autoantibodies against citrullinated proteins in rheumatoid arthritis; anti-nuclear antibodies and serum complement levels in SLE) can also be used to assess reduction in disease and are known in the art. See Shapiro, Cureus. 13(5): e15063 (2021) and Yu et al., Biomolecules. 11(7):928 (2021).

In some embodiments, the method for treating an autoimmune disease in a subject in need thereof with the conjugates of the present invention results in an alteration or impairment in the function of IGF-1R-expressing cells in said subject. As used herein, alteration or impairment refers to any changes to the endogenous function of said IGF-1R-expressing cells in the autoimmune disease.

In some embodiments, the method for treating an autoimmune disease in a subject in need thereof with the conjugates of the present invention results in a reduction in the number of IGF-1R-expressing cells in the subject. In a specific embodiment, the reduction is caused by killing of IGF-1R-expressing cells. Without being bound by theory, it is believed that simple inhibition of the IGF-1R pathway may be insufficient as a fully curative treatment due to signaling by other, potentially redundant, pathways that can continue to act as disease effectors or by compensatory mechanisms or pathways that are upregulated and can lead to adaptive resistance. Thus, the targeted delivery of an agent that is disease-modifying, including one that results in the killing of IGF-1R-expressing cells, may provide benefit beyond (including being preferential over) simple IGF-1R pathway inhibition, as it bypasses the effects of all such potentially redundant and/or compensatory pathways.

In a specific embodiment, the IGF-1R-expressing cells are B lymphocytes and/or T lymphocytes. Autoreactive B lymphocytes can produce pathogenic autoantibodies that can cause tissue damage and/or dysregulate cellular signaling, whereas autoreactive T lymphocytes can activate autoreactive B cells and contribute to inflammation and tissue damage.

In some embodiments, the IGF-1R is overexpressed by cells in the subject diagnosed with an autoimmune disease relative to cells from a healthy subject or a subject that has not been diagnosed with an autoimmune disease. In some embodiments, the frequency of cells expressing IGF-1R in the subject diagnosed with autoimmune disease is increased relative to cells from a healthy subject or a subject that has not been diagnosed with an autoimmune disease. In some embodiments, the frequency of IGF-1R-expressing cells is measured by flow cytometry or immunohistochemistry using techniques and cell surface markers that are known in the art.

In certain embodiments, the reduction in the number of IGF-1R-expressing cells obtained through the methods of the present invention results in amelioration of disease symptoms or reduction in disease. Thus, also provided herein is a method for using modulation in the frequency of IGF-1R-expressing cells to monitor the effectiveness of a treatment of an autoimmune disease in a subject in need thereof, the method comprising administering to the subject an effective amount of a conjugate, wherein said conjugate comprises (i) an IGF-1R ligand, or portion or variant thereof, and (ii) a disease-modifying agent, and subsequently determining the frequency of IGF-1R-expressing cells in a sample obtained from the subject. For example, the treatment can reduce the frequency of IGF-1R-expressing cells in the subject by at least 5%, 10%, 15%, 20%, 25%, 30%, 33%, 35%, 40%, 45%, 50%, 55%, 60%, 70%, 75%, 80%, 85%, 90%, 91%, 92%, 93%, 94%, 95%, 96%, 97%, 98%, 99% or more as compared to the frequency of IGF-1R-expressing cells in the subject before undergoing treatment with the conjugates of the present invention. In certain embodiments, the frequency of IGF-1R-expressing cells in the subject is reduced by about 20% to about 95% compared to the level before initiation of treatment. For example, in certain embodiments, the frequency of IGF-1R-expressing cells in the subject in reduced by about 20%, about 30%, about 40%, about 50%, about 60%, about 70%, about 80%, or about 90% compared to the level before initiation of treatment.

In certain embodiments, the sample used to determine the frequency of IGF-1R-expressing cells is a peripheral blood mononuclear cell (PBMC) sample. Methods of PBMC isolation are routine in the art.

### Combination Treatments

In some embodiments, the conjugate of the present invention is administered in combination with one or more other therapies. Any therapy (e.g., therapeutic or prophylactic agent) that is U.S. Food and Drug Administration (FDA) approved, useful, has been used, or is currently being used for the treatment and/or management of an autoimmune disease can be used in combination with the conjugate of the present invention, including: including: prednisone, hydroxychloroquine, chloroquine, belimumab, anifrolumab, abatacept, atacicept, lupuzor, rituximab, voclosporin, aldesleukin, baricitinib, BIIB059, BI655064, bortezomib, BT063, cenerimod, dapirolizumab pegol, edratide, filgotinib, GS-9876, iberdomide, IFN-α kinoid, iguratimod, nelfinavir, obinutuzumab, OMS721, rapamycin, RC18, RSLV-132, SM101, theralizumab, ustekinumab, vobarilizumab, XmAb5871, blisibimod, tabalumab, epratuzumab, rigerimod, tacrolimus, rontalizumab, sifalimumab, anifrolumab, tocilizumab, infliximab, metelimumab, fresolimumab, rilonacept, cyclophosphamide, methotrexate, nintedanib, JBT-101, imatinib, pirfenidone, nilotinib, dasatinib, SAR100842, BMS-986202, BAY41-2272, riociguat, resunab, ixekizumab, brodalumab, tralokinumab, etanercept, adalimumab, golimumab, secukinumab, tildrakizumab, tofacitinib, and guselkumab.

In some embodiments, the combination of agents disclosed herein allows one or more agents to be dosed at a lower dosage level than the dose at which said agent would have an effect when dosed as a single agent.

### Dosing and Administration

The agents of this invention may be dosed at any clinically relevant dose. By clinically relevant, it is meant that the dose of the agent has an effect in the subject.

In some embodiments, the conjugate of the present invention is administered at a dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, or 10.0 μEq/kg of body weight, where a μEq is equivalent to a mmol of chemotherapeutic agent groups conjugated to the IGF-1R ligand.

In some embodiments, the conjugate of the present invention is administered at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 μEq/kg of body weight, where a μEq is equivalent to a mmol of chemotherapeutic agent groups conjugated to the IGF-1R ligand.

In some embodiments, the conjugate of the present invention is administered at a dose of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, or 16.5 mg/kg of body weight, where a mg refers to the amount of IGF-1R ligand present in the conjugate.

In some embodiments, the conjugate of the present invention is administered at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight, where a mg refers to the amount of IGF-1R ligand present in the conjugate.

In some embodiments, the conjugate of the present invention is dosed at the maximum tolerated dose (MTD). “MTD,” as used herein, refers to the highest dose of an agent that an individual patient can tolerate. In other words, side effects in a given patient can determine the MTD. Side effects may limit the ability to administer higher doses of a treatment than the maximum tolerated dose. Therefore, the MTD for a given patient may be lower than those indicated in the prescribing information for the treatment or those commonly used in clinical practice. The MTD may have limited or no clinical efficacy in a patient.

In some embodiments, the conjugate of the present invention is administered daily, every other day, every three days, every four days, every five days, every six days, once per week, once every two weeks, once every three weeks, once every four weeks, once per month, every two months, or every three months.

The conjugate of the present invention can be administered by any convenient route, for example, by infusion or bolus injection, by absorption through epithelial or mucocutaneous linings (e.g., oral mucosa, rectal, and intestinal mucosa, etc.). Administration can be systemic or local. Various delivery systems are known, e.g., microparticles, microcapsules, capsules, etc., and may be useful for administering a composition of the invention. Methods of administration may include, but are not limited to, oral administration and parenteral administration; parenteral administration including, but not limited to, intradermal, intramuscular, intraperitoneal, intravenous, subcutaneous; intranasal, epidural, sublingual, intracerebral, intraarterial, intraventricular, intrathecal, intravaginal, intracapsular, intraorbital, intracardiac, intradermal, transtracheal, subcuticular, subcapsular, subarachnoidal, intraspinal, intra-cerebrospinal, intrasternal, transdermal, rectally, vaginal, by inhalation, or topically to the ears, nose, eyes, or skin.

The mode of administration is left to the discretion of the practitioner, and will depend, in part, upon the site of the medical condition.

In some embodiments, the compositions of the invention are administered parenterally. In some embodiments, the compositions of the invention are administered intravenously. In another embodiment, the compositions of the invention are administered by continuous infusion. In a particular embodiment, the compositions of the invention are administered by an infusion that lasts for 15 minutes, 20 minutes, 30 minutes, 45 minutes, 1 hour, or 2 hours.

In some embodiments, it can be desirable to administer one or more compositions of the invention locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as sialastic membranes, or fibers. In certain embodiments, one or more compositions of the invention can be injected intraperitoneally.

In yet another embodiment, the compositions of the invention can be delivered in a controlled release system.

In some embodiments, a pump can be used to deliver the compositions of the invention (see, e.g, Sefton, CRC Crit. Ref. Biomed. Eng. 1987, 14, 201; Buchwald et al., Surgery 1980, 88: 507; Saudek et al., N. Engl. J. Med. 1989, 321:574). In some embodiments, the pump may be, but is not limited to, an insulin-like pump.

In some embodiments, the conjugate of the present invention is administered orally, intravenously, subcutaneously, intramuscularly, or topically.

In some embodiments, the method of the present invention does not cause unacceptable hyperglycemia in the subject. Hyperglycemia is another term for high blood glucose and can occur when there is insufficient insulin in the body or when the body cannot utilize insulin properly. Unacceptable hyperglycemia refers to an adverse effect of grade 3 or higher, as determined by a treating physician, and/or one that cannot be controlled with diabetic medications and leads to discontinuation of treatment with the conjugate of the present invention.

### Patient Populations

In some embodiments, the subject having an autoimmune disease treated in accordance with the methods described herein has not previously received treatment for the autoimmune disease. In some embodiments, the subject having an autoimmune disease treated in accordance with the methods described herein has previously received treatment for the autoimmune disease.

In some embodiments, the subject was refractory to previous treatment for the autoimmune disease. As used herein, a subject is “refractory” to prior treatment if the subject has failed to achieve a response to a therapy such that the therapy is determined to not be therapeutically effective, such as: failure to reach clinical endpoint, including any of response, extended duration of response, extended disease-free survival, relapse-free survival, progression-free survival, and overall survival.

The subject matter described herein includes, but is not limited to, the following embodiments:
1. A method for treating an autoimmune disease in a subject, said method comprising administering to the subject an effective amount of a conjugate, wherein said conjugate comprises (i) an insulin-like growth factor 1 receptor (IGF-1R) ligand, or portion or variant thereof, and (ii) a disease-modifying agent.
2. The method of embodiment 1, wherein said autoimmune disease is selected from: adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, Cushing's syndrome, cutaneous graft-versus-host disease (GVHD), dense deposit disease, dermatomyositis, essential mixed cryoglobulinemia, fibromyalgia-fibromyositis, glomerulonephritis (e.g., IgA nephropathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, inflammatory arthritis, irritable bowel disease, juvenile arthritis, lichen planus, lichen sclerosus, lupus [e.g., systemic lupus erythematosus (SLE), cutaneous lupus, discoid lupus), Ménière's disease, mixed connective tissue disease, morphea, multiple sclerosis, Myasthenia Gravis, myocarditis, type 1 or immune-mediated diabetes mellitus, neuritis, other endocrine gland failure, pemphigus vulgaris, pernicious anemia, polyarteritis nodosa, polychondritis, polyendocrinopathies, polyglandular syndromes, polymyalgia rheumatica, polymyositis, post-MI, primary agammaglobulinemia, primary biliary cirrhosis, psoriasis, psoriatic arthritis, Raynaud's phenomenon, relapsing polychondritis, Reiter's syndrome, rheumatic heart disease, rheumatoid arthritis, sarcoidosis, Sjogren's syndrome, stiff-man syndrome, systemic sclerosis (SSc), takayasu arteritis, temporal arteritis/giant cell arteritis, ulcerative colitis, urticaria, uveitis, Uveitis Ophthalmia, vasculitides such as dermatitis herpetiformis vasculitis, vitiligo, or Wegener's granulomatosis.
3. The method of embodiment 2, wherein the autoimmune disease is rheumatoid arthritis, Graves' disease, SSc, Cushing's syndrome, idiopathic pulmonary fibrosis, SLE, or Crohn's disease.
4. The method of embodiment 3, wherein the SSc is diffuse cutaneous SSc.
5. The method of any one of embodiments 1-4, wherein said treatment results in an alteration or impairment in the function of IGF-1R-expressing cells in said subject.
6. The method of any one of embodiments 1-4, wherein said treatment results in a reduction in the number of IGF-1R-expressing cells of said subject.
7. The method of claim embodiments 6, wherein said reduction is caused by killing of said IGF-1R-expressing cells.
8. The method of any one of embodiments 5-7, wherein said IGF-1R-expressing cells are B lymphocytes and/or T lymphocytes.
9. The method of any one of embodiments 1-8, wherein the IGF-1R is overexpressed by cells in said subject relative to cells from a healthy subject or a subject that has not been diagnosed with said autoimmune disease.
10. The method of any one of embodiments 1-9, wherein the frequency of cells expressing IGF-1R in said subject is increased relative to cells from a healthy subject or a subject that has not been diagnosed with said autoimmune disease.
11. The method of embodiment 10, wherein said frequency of IGF-1R-expressing cells is measured by flow cytometry or immunohistochemistry.
12. The method of any one of embodiments 1-11, wherein said treatment results in disease modification in said subject.
13. The method of any one of embodiments 1-12, wherein said IGF-1R ligand comprises wildtype insulin-like growth factor 1 (IGF-1) (SEQ ID NO:3), wildtype insulin (SEQ ID NO:10 and SEQ ID NO:11), or wildtype insulin-like growth factor 2 (IGF-2) (SEQ ID NO:12).
14. The method of any one of embodiments 1-12, wherein said IGF-1R ligand comprises a variant of wildtype IGF-1 (SEQ ID NO:3), a variant of wildtype insulin (SEQ ID NO:10 and SEQ ID NO:11), or a variant of wildtype IGF-2 (SEQ ID NO:12).
15. The method of embodiment 14, wherein said variant of wildtype IGF-1 is at least 90% identical to IGF-1 (SEQ ID NO:3), said variant of wildtype insulin is at least 90% identical to insulin (SEQ ID NO:10 and SEQ ID NO:11), or said variant of wildtype IGF-2 is at least 90% identical to IGF-2 (SEQ ID NO:12).
16. The method of embodiment 14 or 15, wherein said variant of IGF-1 has reduced binding affinity for insulin-like growth factor binding proteins (IGFBPs) as compared to wildtype IGF-1 (SEQ ID NO:3) or said variant of IGF-2 has reduced binding affinity for IGFBPs as compared to wildtype IGF-2 (SEQ ID NO:12).
17. The method of any one of embodiments 14-16, wherein said variant of IGF-1 has increased affinity for the IGF-1R than wildtype IGF-1 (SEQ ID NO:3) or said variant of IGF-2 has increased affinity for the IGF-1R than wildtype IGF-2 (SEQ ID NO:12).
18. The method of any one of embodiments 1-12 or embodiments 14-17, wherein said IGF-1R ligand comprises 765IGF (SEQ ID NO:2), IGF-132 (SEQ ID NO:4), long-R3-IGF-1 (SEQ ID NO:5), R3-IGF-1 (SEQ ID NO:6), des (1-3)-IGF-1 (SEQ ID NO:7), long-IGF-1 (SEQ ID NO:8), or long-G3-IGF-1 (SEQ ID NO:9).
19. The method of any one of embodiments 1-18, wherein said IGF-1R ligand, or portion or variant thereof, comprises a leader sequence.
20. The method of embodiment 19, wherein said leader sequence comprises SEQ ID NO:1.
21. The method of any one of embodiments 18-20, wherein said IGF-1R ligand comprises 765IGF (SEQ ID NO:2).
22. The method of any one of embodiments 1-21, wherein said disease-modifying agent comprises a cytotoxic agent.
23. The method of embodiment 22, wherein said cytotoxic agent comprises a chemotherapeutic agent.
24. The method of embodiment 23, wherein said chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine.
25. The method of embodiment 24, wherein said chemotherapeutic agent is methotrexate.
26. The method of embodiment 22, wherein said cytotoxic agent comprises a toxin.
27. The method of embodiment 26, wherein said toxin comprises Clostridium perfringens enterotoxin, diphtheria toxin, ricin chain A, Pseudomonas exotoxin, A chain toxins, a ribosome inactivating protein, α-sarcin, aspergillin, or a ribonuclease.
28. The method of embodiment 27, wherein said toxin comprises Clostridium perfringens enterotoxin, or a portion or variant thereof.
29. The method of embodiment 28, wherein the conjugate comprises SEQ ID NO:14 or SEQ ID NO:15.
30. The method of embodiment 27, wherein said toxin comprises diphtheria toxin, or a portion or variant thereof.
31. The method of embodiment 30, wherein the conjugate comprises SEQ ID NO:13 or SEQ ID NO:16.
32. The method of any one of embodiments 1-31, wherein said subject (i) has not previously received treatment for said autoimmune disease; (ii) has previously received treatment for said autoimmune disease; (iii) has relapsed from previous treatment for said autoimmune disease; or (iv) was refractory to previous treatment for said autoimmune disease.
33. The method of any one of embodiments 1-32, wherein said conjugate is administered in combination with one or more other therapies.
34. The method of embodiment 33, wherein said one or more other therapies comprises one or more of the following: prednisone, hydroxychloroquine, chloroquine, belimumab, anifrolumab, abatacept, atacicept, lupuzor, rituximab, voclosporin, aldesleukin, baricitinib, BIIB059, BI655064, bortezomib, BT063, cenerimod, dapirolizumab pegol, edratide, filgotinib, GS-9876, iberdomide, IFN-a kinoid, iguratimod, nelfinavir, obinutuzumab, OMS721, rapamycin, RC18, RSLV-132, SM101, theralizumab, ustekinumab, vobarilizumab, XmAb5871, blisibimod, tabalumab, epratuzumab, rigerimod, tacrolimus, rontalizumab, sifalimumab, anifrolumab, tocilizumab, infliximab, metelimumab, fresolimumab, rilonacept, cyclophosphamide, methotrexate, nintedanib, JBT-101, imatinib, pirfenidone, nilotinib, dasatinib, SAR100842, BMS-986202,BAY41-2272, riociguat, resunab, ixekizumab, brodalumab, tralokinumab, etanercept, adalimumab, golimumab, secukinumab, tildrakizumab, tofacitinib, and guselkumab.
35. The method of any one of embodiments 1-25, wherein said conjugate is administered at dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, 9.5, or 10.0 μEq/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 μEq/kg of body weight.
36. The method of any one of embodiments 1-34, wherein said conjugate is administered at dose of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, or 16.5 mg/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight.
36. The method of any one of embodiments 1-34, wherein said conjugate is administered at dose that is the maximum tolerated dose.
37. The method of any one of embodiments 1-37, wherein said conjugate is administered daily, every other day, every three days, every four days, every five days, every six days, once per week, once every two weeks, once every three weeks, once every four weeks, once per month, every two months, or every three months.
38. The method of any one of embodiments 1-38, wherein said conjugate is administered orally, intravenously, subcutaneously, intramuscularly, or topically.
39. The method of any one of embodiments 1-39, wherein said method does not cause unacceptable hyperglycemia in said subject.
40. A method for treating an autoimmune disease in a subject, said method comprising administering to the subject an effective amount of a conjugate, wherein said conjugate comprises (i) 765IGF (SEQ ID NO:2) and (ii) methotrexate.
41. A method for using modulation in the frequency of IGF-1R-expressing cells to monitor the effectiveness of a treatment of an autoimmune disease in a subject in need thereof, the method comprising:
   a) administering to the subject an effective amount of a conjugate, wherein said conjugate comprises (i) an IGF-1R ligand, or portion or variant thereof, and (ii) a disease-modifying agent; and
   b) subsequently determining the frequency of IGF-1R-expressing cells in a sample obtained from the subject.

The following examples are offered by way of illustration and not by way of limitation.

### EXAMPLES

### Example 1: Effect of LX-101 on Viability and Phenotype of PBMCs From Patients With Autoimmune Diseases

LX-101 is an IGF-1R ligand (SEQ ID NO: 2) conjugate with methotrexate, a well-known inhibitor of dihydrofolate reductase, which ultimately inhibits nucleotide synthesis. Without wishing to be bound by theory, LX-101 may deliver methotrexate to cells overexpressing IGF-1R implicated in the pathology of autoimmune diseases and reduce their viability and proliferation.

Frozen peripheral blood mononuclear cells (PBMCs) were obtained from Discovery Life Sciences from patients diagnosed with lupus, rheumatoid arthritis, and scleroderma. One sample was obtained from a 74-year-old white female diagnosed with systemic lupus erythematosus (SLE) undergoing active treatment with methotrexate, Celebrex, prednisone, and Benlysta. A second sample was obtained from a 46-year-old white female diagnosed with scleroderma (SSc) before receiving treatment. A third sample was obtained from a 71-year-old white female diagnosed with rheumatoid arthritis (RA) undergoing active treatment with Nabumetone. All samples were collected following informed consent.

The PBMCs were thawed, washed, and resuspended in AIM V media with 5% human AB serum (hAB) at a concentration of 1×10⁶ cells/mL. PBMCs were seeded in a 24 well plate (1×10⁶ cells/well) and treated with a 17 μM of LX-101 or hydrochloric acid (HCl) as a vehicle control. LX-101 was supplied at a concentration of 4 mM (where the concentration is expressed in terms of the amount of methotrexate in the drug) in 10 mM HC1. The vehicle control groups received the same concentration of HCl as that in the LX-101 solution received by the treatment group. PBMCs were incubated at 37° C. with 5% CO₂ for three days.

After the three day incubation with LX-101 or the vehicle control, the PBMCs were blocked with human TruStain FcX Fc receptor blocking solution, and stained with an antibody mixture with markers shown in Table 2. Cells were fixed and flow cytometric data were collected for 50,000 live cell events using a BD LSRFortessa X-20 flow cytometer. Data were analyzed with Kaluza software.

TABLE 2
Flow Cytometry Antibodies

| Marker | Fluorochrome | Clone | Catalog # | Vendor |
|---|---|---|---|---|
| CD3 | BUV395 | SK7 | 564001 | BD |
| CD4 | BV421 | OKT4 | 317434 | Biolegend |
| CD8 | FITC | SK1 | 344704 | Biolegend |
| CD19 | BV711 | HIB19 | 302245 | Biolegend |
| CD14 | APC | HCD14 | 325607 | Biolegend |
| CD56 | BV711 | HCD56 | 318335 | Biolegend |
| CD11C | PE-Cy7 | 3.9 | 301607 | Biolegend |
| CD123 | BV605 | 6H6 | 306025 | Biolegend |
| IGF-1R/CD221 | PE | 1H7 | 555999 | BD |
| Live/Dead | APC-eFluor780 | N/A | 65-0865-14 | eBiosciences |

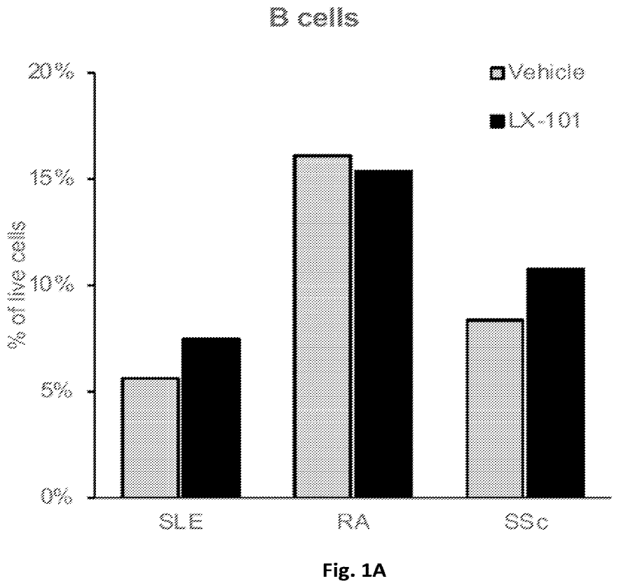

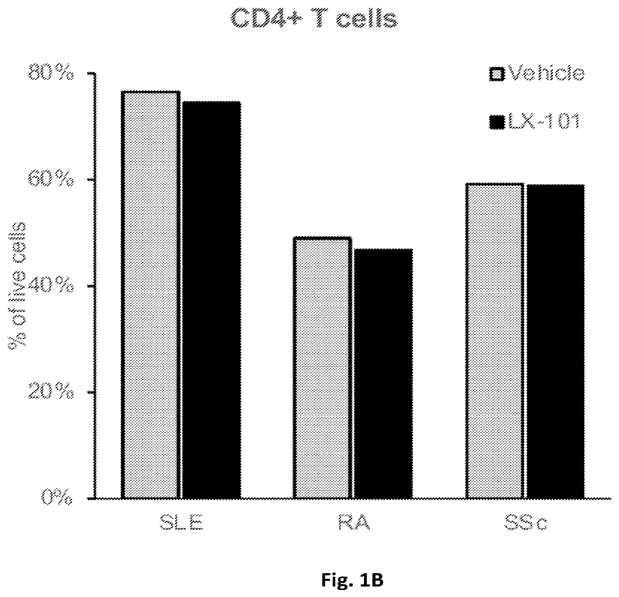

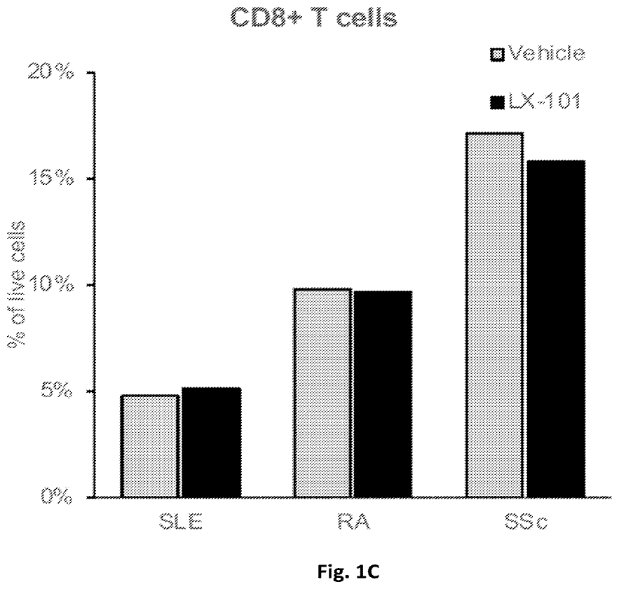

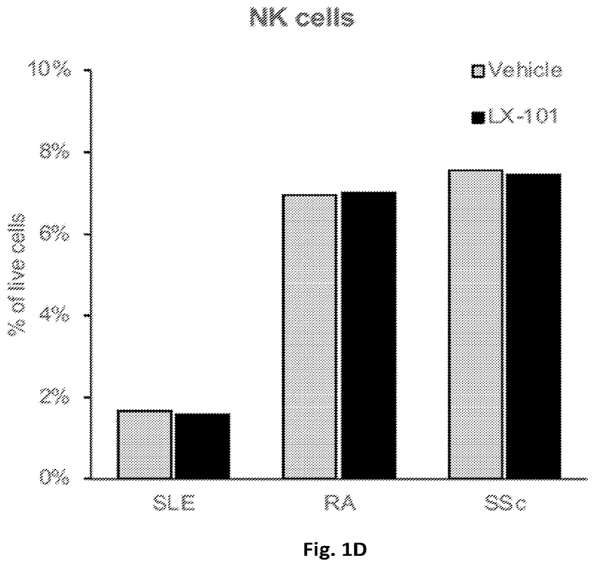

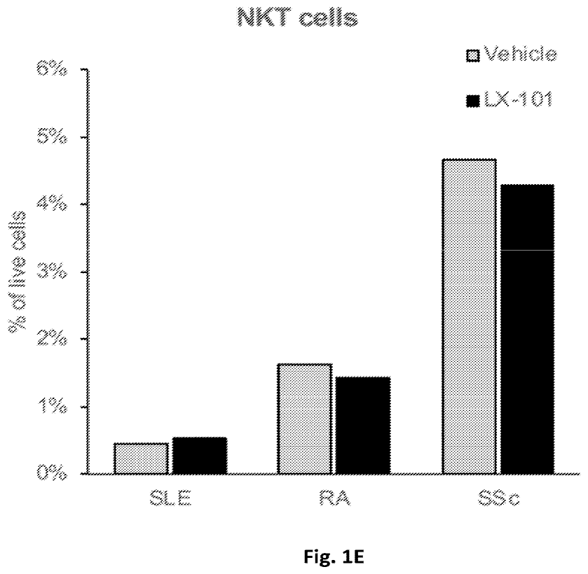

The effect of LX-101 was analyzed for both the bulk PBMC and IGF-1R⁺ subpopulations. The percentages of live, B cells, CD4⁺ T cells, CD8⁺ T cells, NK cells, and NKT cells were determined. Little difference was observed between the vehicle control and LX-101 treated cells for samples from SLE, RA and SSc patients for the bulk population of PBMCs (FIGS. 1A-1E).

However, for the IGF-1R⁺ cells, marked reductions of subpopulations were observed for the LX-101 treated cells as compared to the vehicle control.

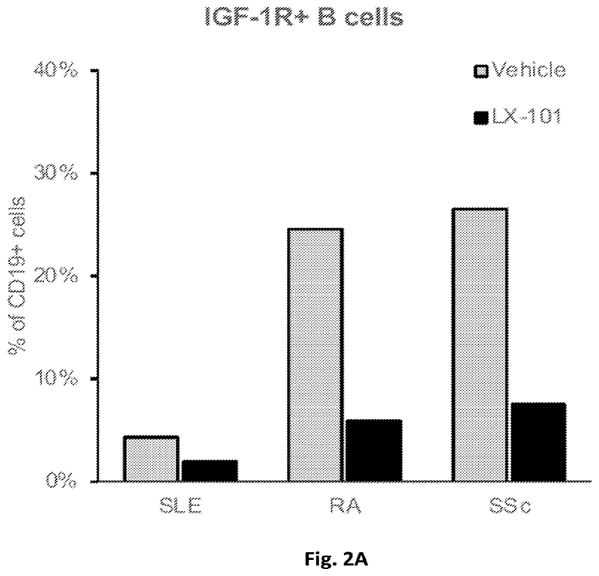

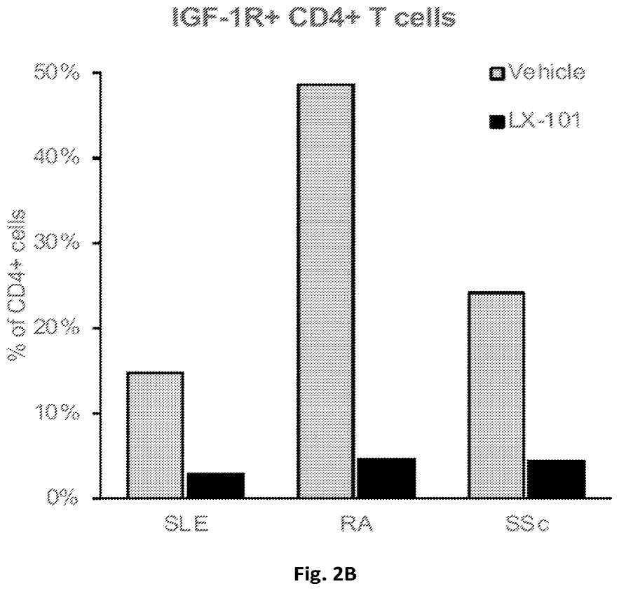

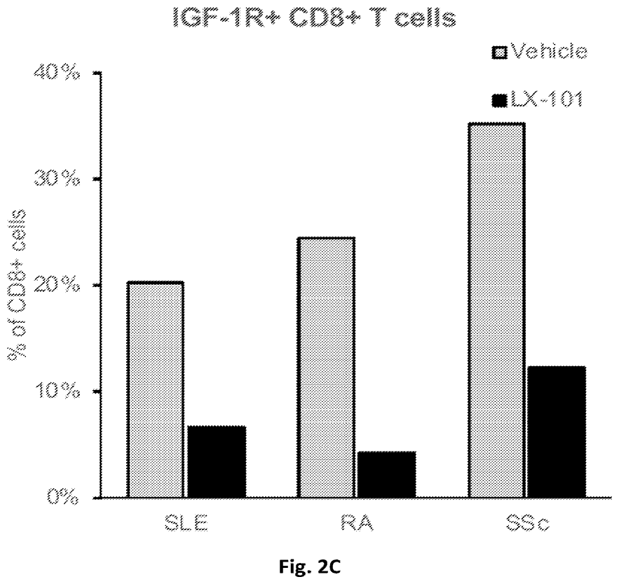

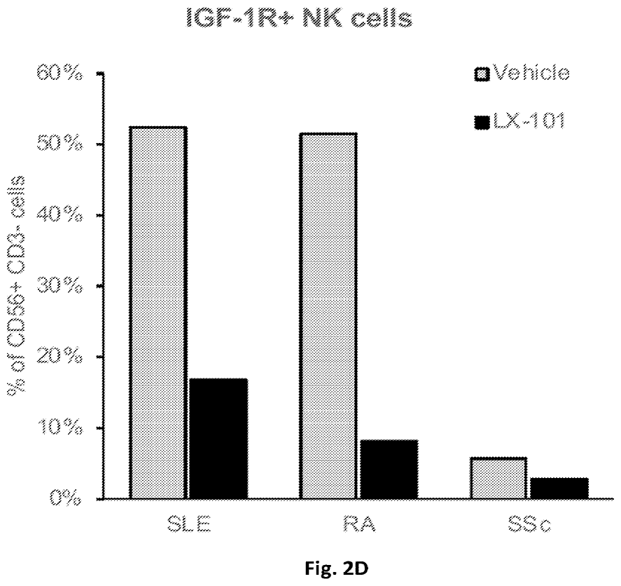

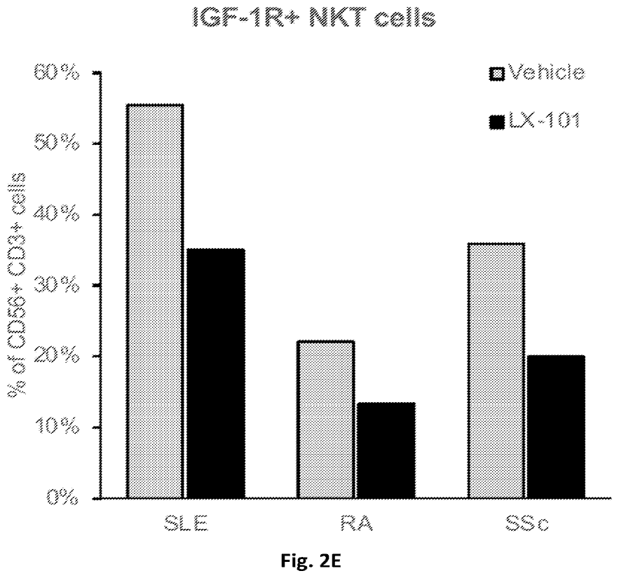

The population of IGF-1R⁺CD19⁺ B cells was also compared, as shown in FIG. 2A. A reduced percentage of IGF-1R⁺CD19⁺ B cells was observed for LX-101 treated patient samples. The SLE sample had a lower B cell population in vehicle control. However, the SLE sample was obtained from a patient being treated with Benlysta (e.g., belimumab), a B lymphocyte stimulator (BLyS)-specific inhibitor, administration of which has been found to result in a decrease in total CD19⁺ B cells in the peripheral blood of SLE patients. However, the number of CD3⁺ T cells was not affected. See Jacobi et al (2010) Arthritis & Rheumatism 62: 201-210.

Both CD4⁺ and CD8⁺ T cell populations were compared for the IGF-1R⁺ populations. A substantial reduction of IGF-1R⁺CD3⁺CD4⁺ T cells was observed for SLE, RA, and SSc samples treated with LX-101, especially for the RA sample which had a high IGF-1R⁺ CD4⁺ T cell expression in the vehicle control (FIG. 2B). A reduction of IGF-1R⁺CD3⁺CD8⁺ T cells was also observed for SLE, RA, and SSc samples treated with LX-101 (FIG. 2C).

As seen in FIG. 2D, IGF-1R⁺CD56⁺CD3⁻ natural killer (NK) cells were reduced in the samples treated with LX-101 as compared to the vehicle control, particularly for samples from SLE and RA patients. Dysregulation of NK cells and NK-derived cytokine production is thought to contribute to the pathology of many autoimmune diseases.

IGF-1R⁺CD56⁺CD3⁺ natural killer T (NKT) cells were compared and a modest reduction of NKT cells was observed for SLE, RA, and SSc samples treated with LX-101, as shown in FIG. 2E.

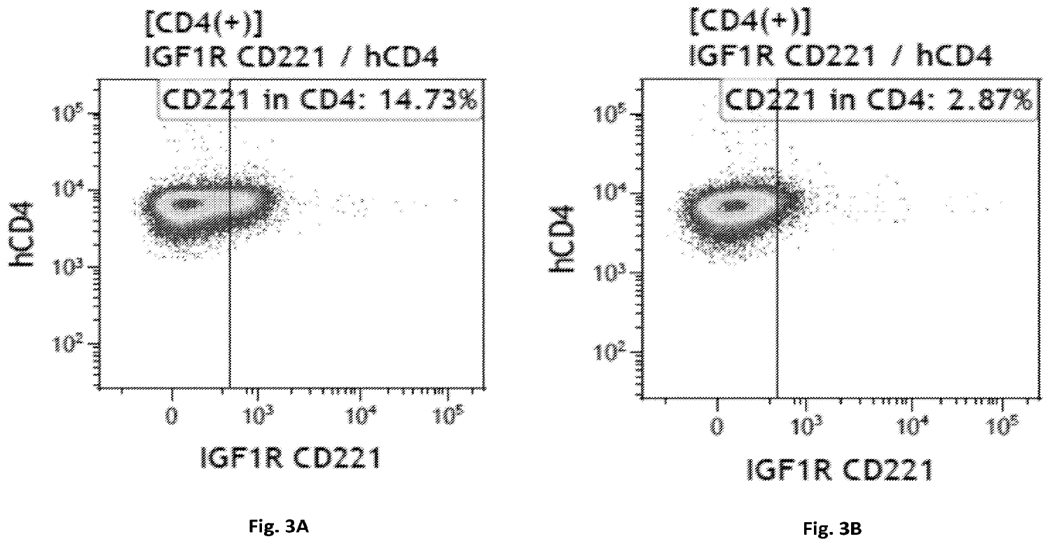

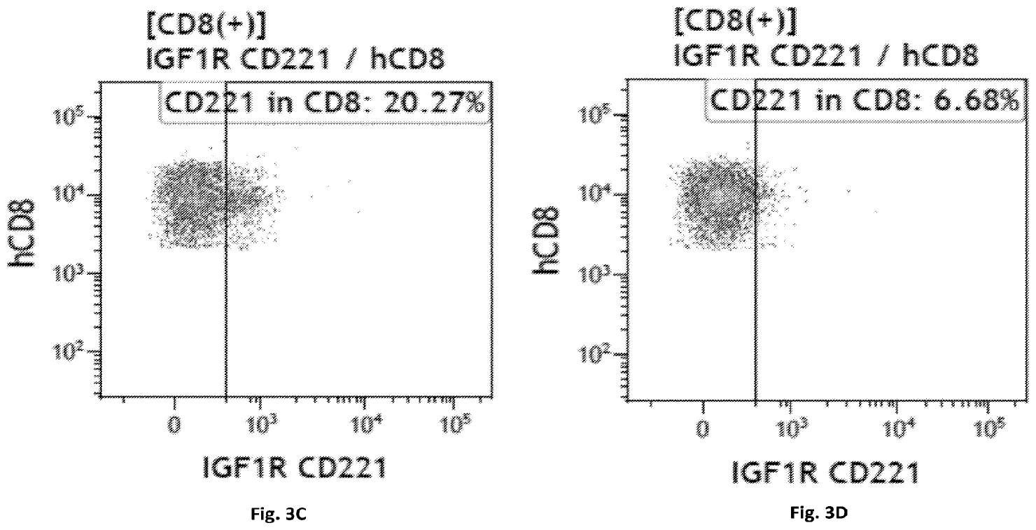

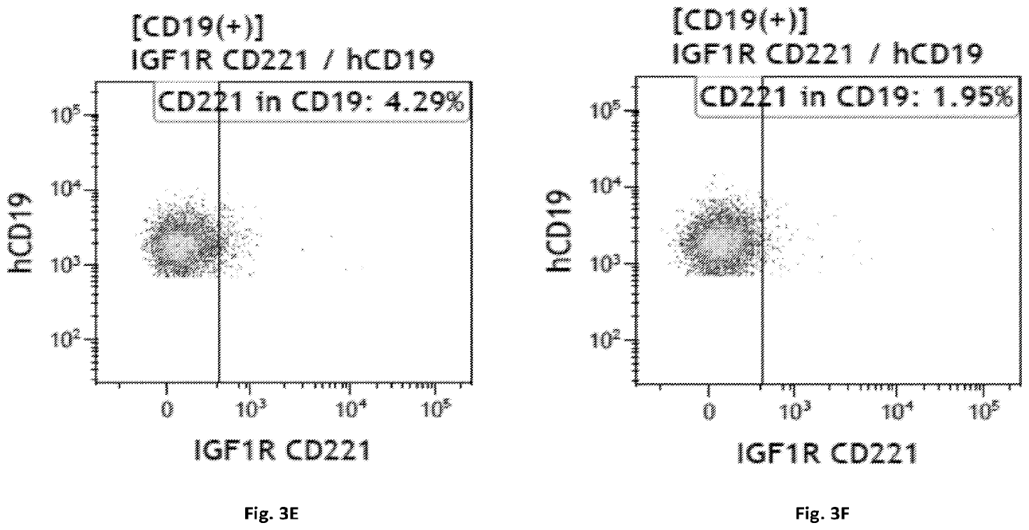

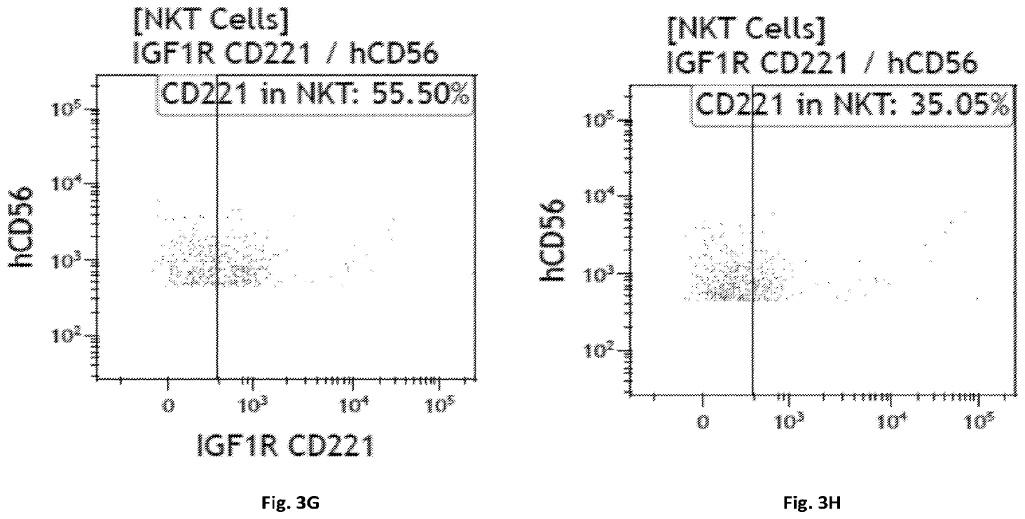

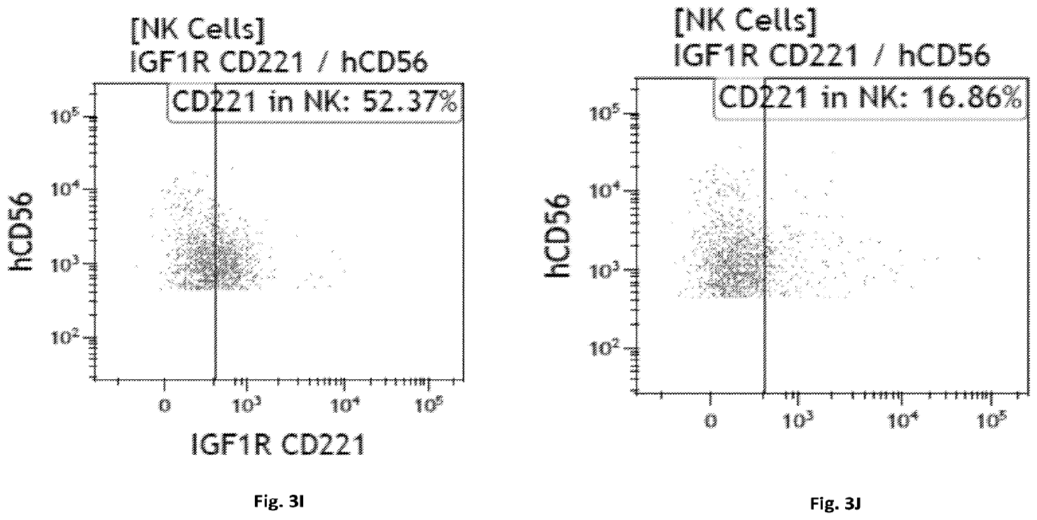

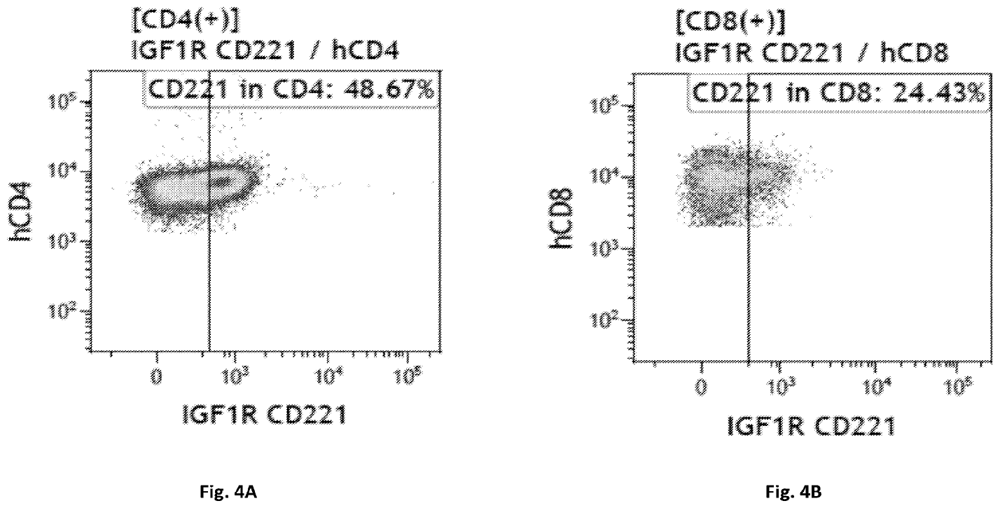

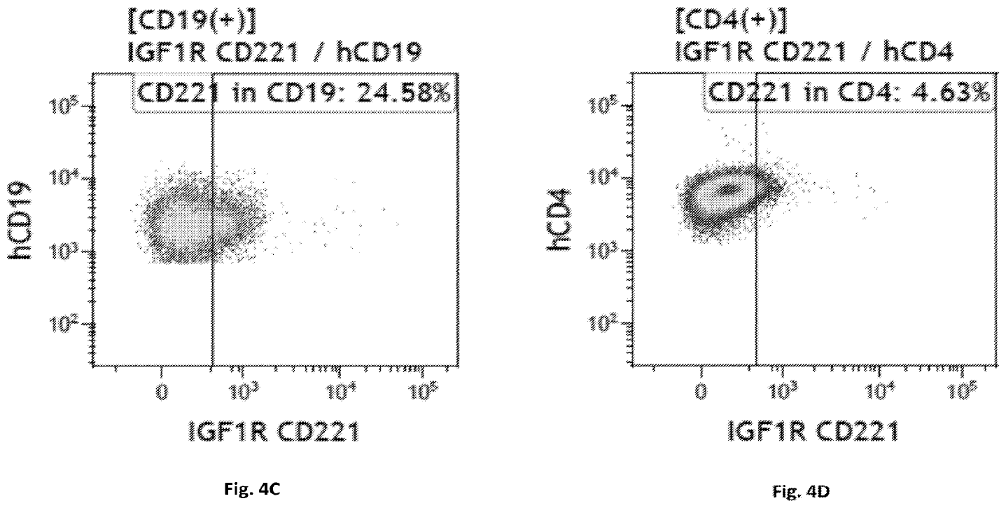

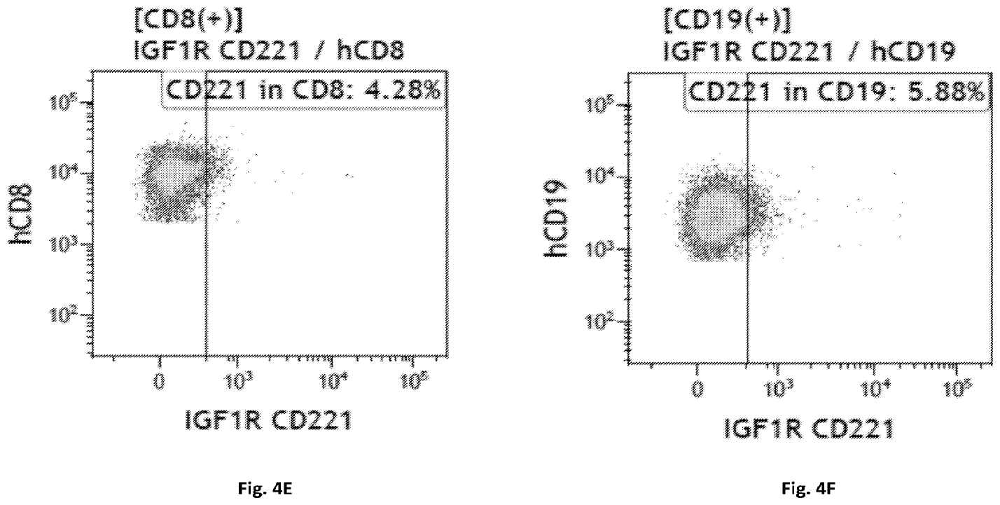

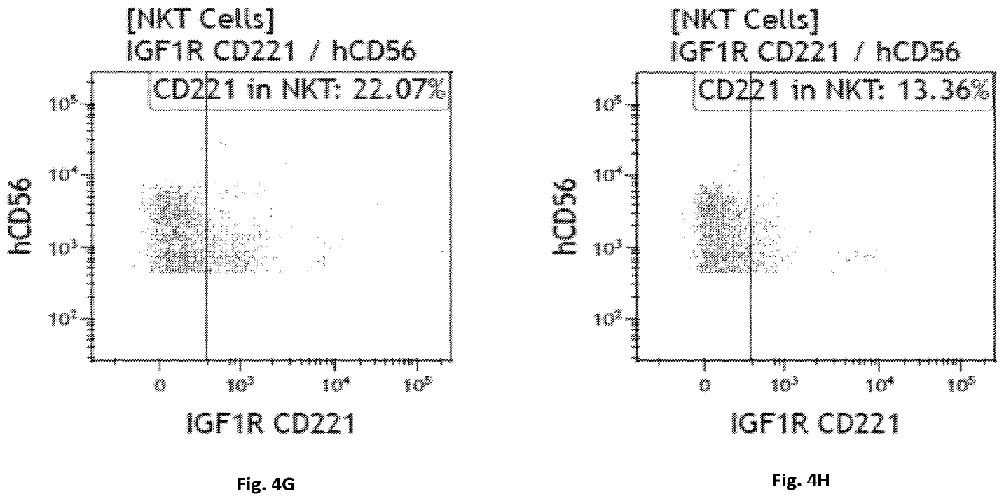

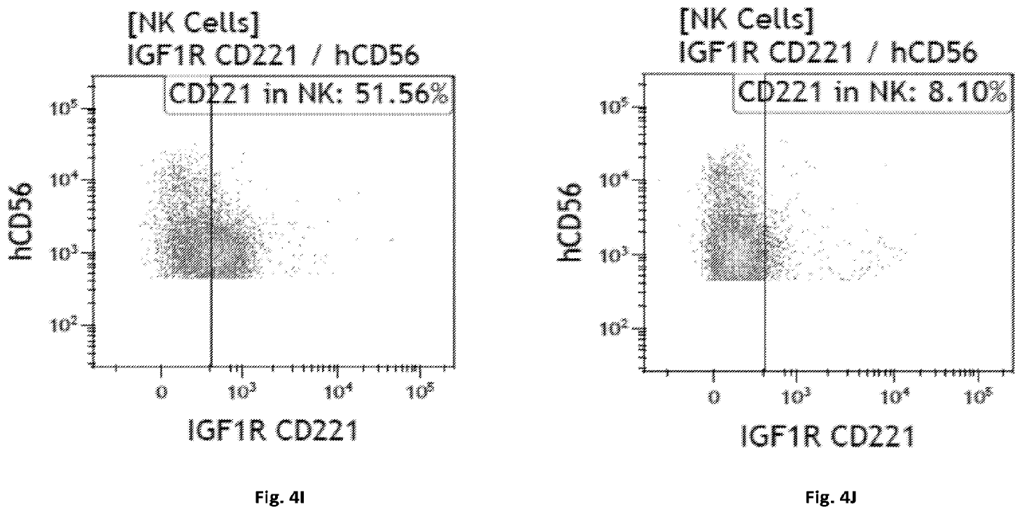

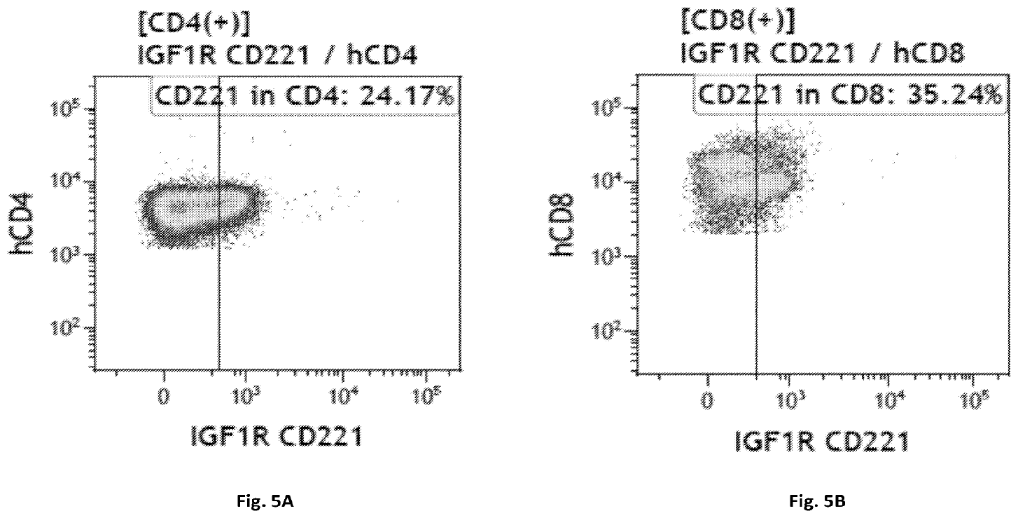

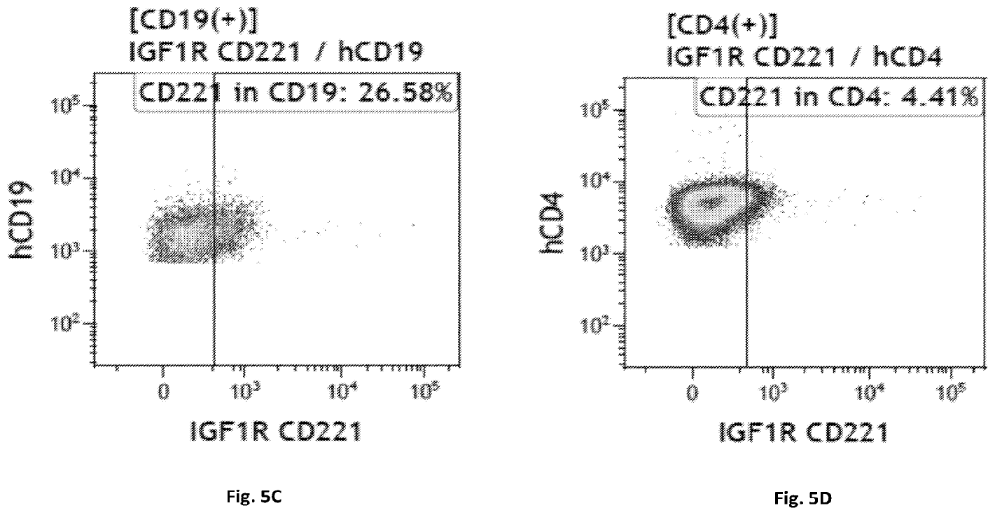

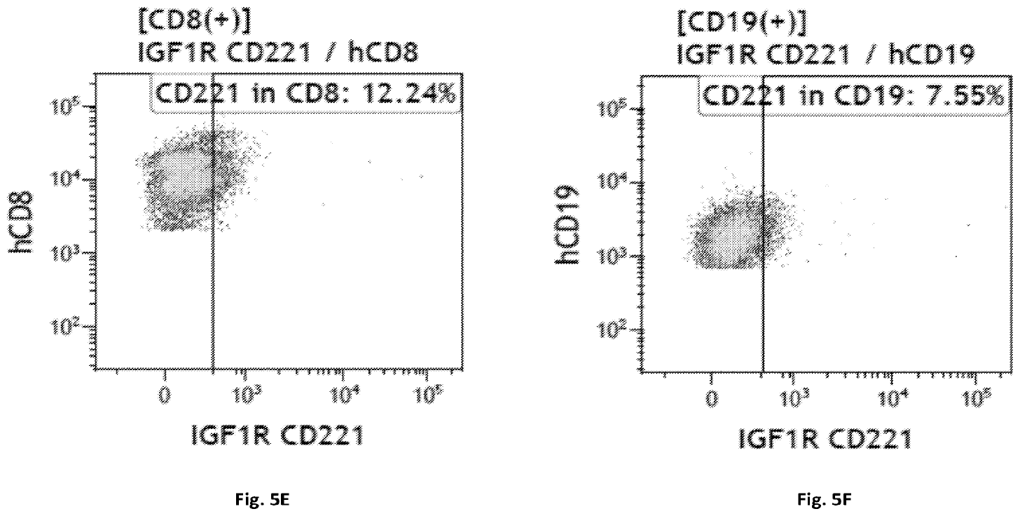

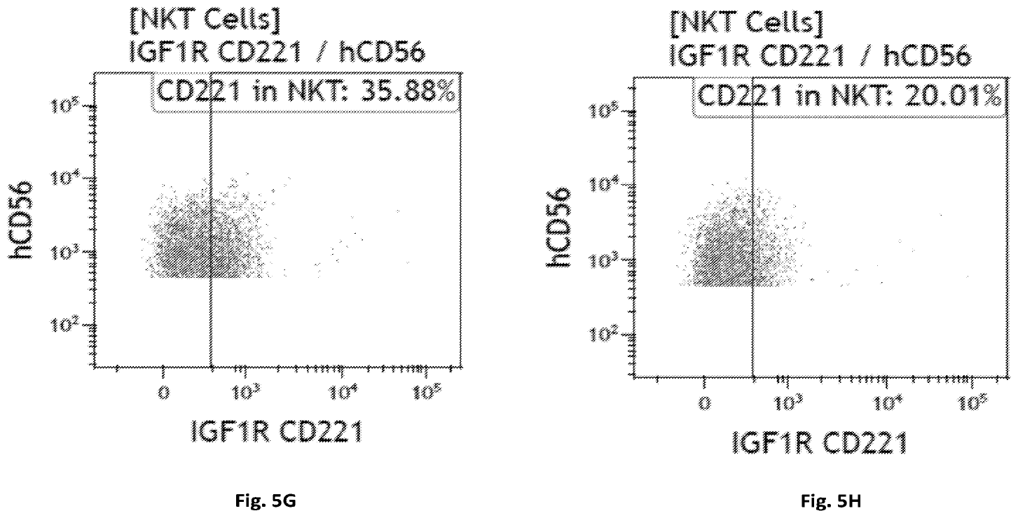

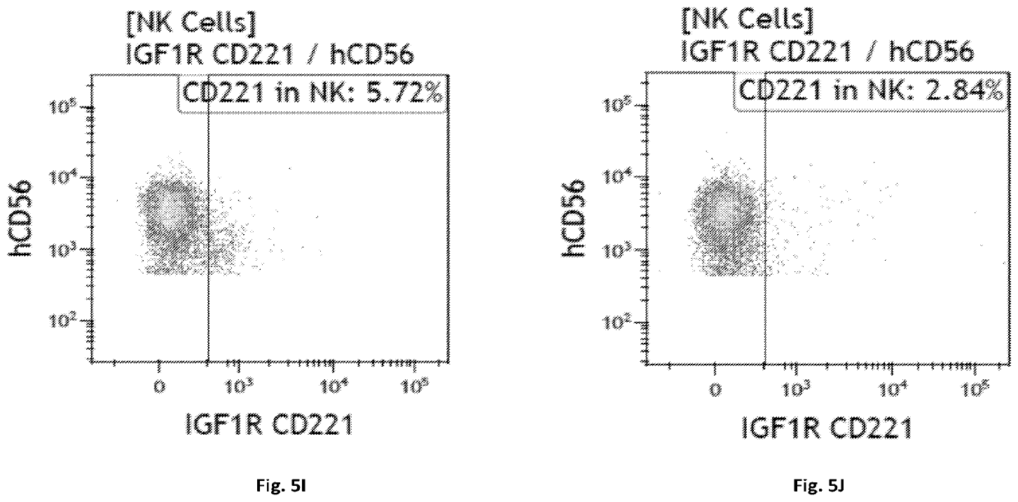

FIGS. 3A-3J show plots of the flow cytometric data for IGF-1R⁺ PBMCs from the SLE patient. A reduction in the IGF-1R⁺ subpopulations of CD4⁻ T cells (FIG. 3A—vehicle control; FIG. 3B—LX-101), CD8⁺ T cells (FIG. 3C—vehicle control; FIG. 3D—LX-101), B cells (FIG. 3E—vehicle control; FIG. 3F—LX-101), NKT cells (FIG. 3G—vehicle control; FIG. 3H—LX-101), and NK cells (FIG. 3I-—ehicle control; FIG. 3J—LX-101) were observed following LX-101 treatment, indicating fewer living cells expressing IGF-1R. The corresponding data from the RA patient are show in FIGS. 4A-4J. The data from the SSc patient are shown in FIGS. 5A-5J.

The data suggest that LX-101 reduces IGF-1R⁺ immune cell populations implicated in the pathogenesis of autoimmune diseases, including SLE, RA, and SSc. Little off-target effects were observed in IGF-1R⁻ cells, and reduced percentages of IGF-1R⁺ B cells, CD4⁺ T cells, CD8⁺ T cells, NK cells, and NKT cells were found in LX-101 treated samples.

Efforts have been made to ensure accuracy with respect to numbers used (e.g., amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical, and scientific terms used herein have the same meaning as commonly understood by one of ordinary skill in the art to which this subject matter belongs.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for treating an autoimmune disease in a subject in need thereof, said method comprising administering to the subject a conjugate comprising an insulin-like growth factor 1 receptor (IGF-1R) ligand, or portion or variant thereof, and a disease-modifying agent.

2. The method of claim 1, wherein said autoimmune disease is selected from the group consisting of adrenergic drug resistance, alopecia areata, ankylosing spondylitis, antiphospholipid syndrome, autoimmune Addison's disease, autoimmune diseases of the adrenal gland, allergic encephalomyelitis, autoimmune hemolytic anemia, autoimmune hepatitis, autoimmune inflammatory eye disease, autoimmune neonatal thrombocytopenia, autoimmune neutropenia, autoimmune oophoritis and orchitis, autoimmune thrombocytopenia, autoimmune thyroiditis, Behcet's disease, bullous pemphigoid, cardiomyopathy, cardiotomy syndrome, celiac sprue-dermatitis, chronic active hepatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, Cushing's syndrome, cutaneous graft-versus-host disease (GVHD), dense deposit disease, dermatomyositis, essential mixed cryoglobulinemia, fibromyalgia-fibromyositis, glomerulonephritis (e.g., IgA nephropathy), gluten-sensitive enteropathy, Goodpasture's syndrome, Graves' disease, Guillain-Barre, hyperthyroidism (i.e., Hashimoto's thyroiditis), idiopathic pulmonary fibrosis, idiopathic Addison's disease, idiopathic thrombocytopenia purpura (ITP), IgA neuropathy, inflammatory arthritis, irritable bowel disease, juvenile arthritis, lichen planus, lichen sclerosus, lupus [e.g., systemic lupus erythematosus (SLE), cutaneous lupus, discoid lupus), Ménière's disease, mixed connective tissue disease, morphea, multiple sclerosis, Myasthenia Gravis, myocarditis, type 1 or immune-mediated diabetes mellitus, neuritis, other endocrine gland failure, pemphigus vulgaris, pernicious anemia, polyarteritis nodosa, polychondritis, polyendocrinopathies, polyglandular syndromes, polymyalgia rheumatica, polymyositis, post-MI, primary agammaglobulinemia, primary biliary cirrhosis, psoriasis, psoriatic arthritis, Raynaud's phenomenon, relapsing polychondritis, Reiter's syndrome, rheumatic heart disease, rheumatoid arthritis, sarcoidosis, Sjogren's syndrome, stiff-man syndrome, systemic sclerosis (SSc), takayasu arteritis, temporal arteritis/giant cell arteritis, ulcerative colitis, urticaria, uveitis, Uveitis Ophthalmia, vasculitides such as dermatitis herpetiformis vasculitis, vitiligo, and Wegener's granulomatosis.

3. The method of claim 2, wherein the autoimmune disease is selected from the group consisting of rheumatoid arthritis, Graves' disease, SSc, Cushing's syndrome, idiopathic pulmonary fibrosis, SLE, and Crohn's disease.

4. The method of claim 3, wherein the SSc is diffuse cutaneous SSc.

5. The method of any one of claims 1-4, wherein said method results in a reduction in a number of IGF-1R-expressing cells of said subject.

6. The method of claim 5, wherein said IGF-1R-expressing cells are selected from the group consisting of B lymphocytes, T lymphocytes, NK cells, and NKT cells.

7. The method of claim 6, wherein said T lymphocytes are CD4⁺ or CD8⁺.

8. The method of claim 5 or 6, wherein said reduction in the number of IGF-1R-expressing cells is measured by flow cytometry or immunohistochemistry.

9. The method of any one of claims 1-8, wherein said IGF-1R ligand comprises wildtype insulin-like growth factor 1 (IGF-1), wildtype insulin, or wildtype insulin-like growth factor 2 (IGF-2).

10. The method of claim 9, wherein said wildtype IGF-1 comprises SEQ ID NO:3, wherein said wildtype insulin comprises SEQ ID NO:10 or 11, and wherein said wildtype IGF-2 comprises SEQ ID NO:12.

11. The method of any one of claims 1-8, wherein said IGF-1R ligand comprises a variant of wildtype IGF-1, a variant of wildtype insulin, or a variant of wildtype IGF-2.

12. The method of claim 11, wherein said variant of wildtype IGF-1 is at least 90% identical to SEQ ID NO:3, said variant of wildtype insulin is at least 90% identical to SEQ ID NO:10 or SEQ ID NO:11, and said variant of wildtype IGF-2 is at least 90% identical to SEQ ID NO:12.

13. The method of claim 11 or 12, wherein (i) said variant of wildtype IGF-1 has reduced binding affinity for insulin-like growth factor binding proteins (IGFBPs) as compared to wildtype IGF-1, or said variant of wildtype IGF-2 has reduced binding affinity for IGFBPs as compared to wildtype IGF-2, and/or (ii) said variant of wildtype IGF-1 has increased affinity for the IGF-1R as compared to wildtype IGF-1 or said variant of wildtype IGF-2 has increased affinity for the IGF-1R as compared to wildtype IGF-2.

14. The method of any one of claims 1-13, wherein said IGF-1R ligand, or portion or variant thereof, comprises a leader sequence.

15. The method of claim 14, wherein said leader sequence comprises SEQ ID NO:1.

16. The method of any one of claims 1-8 and claims 11-15, wherein said IGF-1R ligand comprises 765IGF (SEQ ID NO:2), IGF-132 (SEQ ID NO:4), long-R3-IGF-1 (SEQ ID NO:5), R3-IGF-1 (SEQ ID NO:6), des (1-3)-IGF-1 (SEQ ID NO:7), long-IGF-1 (SEQ ID NO:8), or long-G3-IGF-1 (SEQ ID NO:9).

17. The method of claim 16, wherein said IGF-1R ligand comprises 765IGF (SEQ ID NO:2).

18. The method of any one of claims 1-17, wherein said disease-modifying agent comprises a cytotoxic agent.

19. The method of claim 18, wherein said cytotoxic agent comprises a chemotherapeutic agent.

20. The method of claim 19, wherein said chemotherapeutic agent is amsacrine, azacytidine, bleomycin, busulfan, capecitabine, carboplatin, carmustine, chlorambucil, cisplatin, cladribine, cyclophosphamide, cytarabine, dactinomycin, daunorubicin, decarbazine, docetaxel, doxorubicin, epirubicin, estramustine, etoposide, floxuridine, fludarabine, fluorouracil, gemcitabine, hexamethylmelamine, idarubicin, ifosfamide, irinotecan, lomustine, mechlorethamine, melphalan, mercaptopurine, methotrexate, mitomycin C, mitotane, mitoxantrone, oxaliplatin, paclitaxel, pemetrexed, pentostatin, plicamycin, procarbazine, ralitrexed, semustine, streptozocin, temozolamide, teniposide, thioguanine, thiotepa, topotecan, trimitrexate, valrubicin, vincristine, vinblastine, vindestine, or vinorelbine.

21. The method of claim 20, wherein said chemotherapeutic agent is methotrexate.

22. The method of claim 18, wherein said cytotoxic agent comprises a toxin.

23. The method of claim 22, wherein said toxin comprises Clostridium perfringens enterotoxin, diphtheria toxin, ricin chain A, Pseudomonas exotoxin, A chain toxins, a ribosome inactivating protein, α-sarcin, aspergillin, or a ribonuclease.

24. The method of claim 23, wherein said toxin comprises Clostridium perfringens enterotoxin, or a portion or variant thereof.

25. The method of claim 24, wherein the conjugate comprises SEQ ID NO:14 or SEQ ID NO:15.

26. The method of claim 23, wherein said toxin comprises diphtheria toxin, or a portion or variant thereof.

27. The method of claim 26, wherein the toxin comprises SEQ ID NO:13 or SEQ ID NO:16.

28. The method of any one of claims 1-27, wherein said subject (i) has not previously received treatment for said autoimmune disease; (ii) has previously received treatment for said autoimmune disease; (iii) has relapsed from previous treatment for said autoimmune disease; or (iv) was refractory to previous treatment for said autoimmune disease.

29. The method of any one of claims 1-28, wherein said conjugate is administered in combination with one or more other therapies.

30. The method of claim 29, wherein said one or more other therapies comprise prednisone, hydroxychloroquine, chloroquine, belimumab, anifrolumab, abatacept, atacicept, lupuzor, rituximab, voclosporin, aldesleukin, baricitinib, BIIB059, BI655064, bortezomib, BT063, cenerimod, dapirolizumab pegol, edratide, filgotinib, GS-9876, iberdomide, IFN-α kinoid, iguratimod, nelfinavir, obinutuzumab, OMS721, rapamycin, RC18, RSLV-132, SM101, theralizumab, ustekinumab, vobarilizumab, XmAb5871, blisibimod, tabalumab, epratuzumab, rigerimod, tacrolimus, rontalizumab, sifalimumab, anifrolumab, tocilizumab, infliximab, metelimumab, fresolimumab, rilonacept, cyclophosphamide, methotrexate, nintedanib, JBT-101, imatinib, pirfenidone, nilotinib, dasatinib, SAR100842, BMS-986202, BAY41-2272, riociguat, resunab, ixekizumab, brodalumab, tralokinumab, etanercept, adalimumab, golimumab, secukinumab, tildrakizumab, tofacitinib, or guselkumab.

31. The method of any one of claims 1-30, wherein said conjugate is administered at dose of about 0.05, 0.10, 0.20, 0.40, 0.80, 1.0, 1.5, 1.6, 2.0, 2.5, 3.0, 3.5, 4.0, 4.5, 5.0, 5.5, 6.0, 6.5, 7.0, 7.5, 8.0, 8.5, 9.0, 9.5, or 10.0 μEq/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, or 9.5-10.0 μEq/kg of body weight.

32. The method of any one of claims 1-31, wherein said conjugate is administered at dose of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, or 16.5 mg/kg of body weight or at a dose range of about 0.05-0.5, 0.5-1.0, 1.0-1.5, 1.5-2.0, 2.0-2.5, 2.5-3.0, 3.0-3.5, 3.5-4.0, 4.0-4.5, 4.5-5.0, 5.0-5.5, 5.5-6.0, 6.0-6.5, 6.5-7.0, 7.0-7.5, 7.5-8.0, 8.0-8.5, 8.5-9.0, 9.0-9.5, 9.5-10.0, 10.0-10.5, 10.5-11.0, 11.0-11.5, 11.5-12.0, 12.0-12.5, 12.5-13.0, 13.0-13.5, 13.5-14.0, 14.0-14.5, 14.5-15.0, 15.0-15.5, 15.5-16.0, or 16.0-16.5 mg/kg of body weight.

33. The method of any one of claims 1-32, wherein said conjugate is administered at a dose that is the maximum tolerated dose.

34. The method of any one of claims 1-33, wherein said conjugate is administered daily, every other day, every three days, every four days, every five days, every six days, once per week, once every two weeks, once every three weeks, once every four weeks, once per month, every two months, or every three months.

35. The method of any one of claims 1-34, wherein said conjugate is administered orally, intravenously, subcutaneously, intramuscularly, or topically.

36. The method of any one of claims 1-8 and 11-35, wherein the IGF-1R ligand is SEQ ID NO:2, the cytotoxic agent is methotrexate, wherein there are 6 to 10 methotrexate molecules for every IGF-1R ligand of SEQ ID NO:2.

37. The method of claim 36, wherein the autoimmune disease is SLE.

38. The method of claim 36, wherein the autoimmune disease is RA.

39. The method of claim 36, wherein the autoimmune disease is SSc.
